(19) United States
(12) Patent Application Publication
(10) Pub. No.: US 2025/0143909 A1
(43) Pub. Date: May 8, 2025

(54) Rotating Mechanism, Portable Neck Supporter and Adjusting Method Thereof

(71) Applicant: Jiangsu Alphay Medical Devices Co., Ltd., Jiangsu (CN)

(72) Inventors: Jiang WANG, Jiangsu (CN); Jinfeng JI, Jiangsu (CN); Kai ZHAO, Jiangsu (CN)

(21) Appl. No.: 17/420,135

(22) PCT Filed: Mar. 19, 2021
(86) PCT No.: PCT/CN2021/081712

§ 371 Date: Sep. 22, 2022

(30) Foreign Application Priority Data
Aug. 17, 2020 (CN) 202010826452.0
Aug. 17, 2020 (CN) 202021722208.1
Aug. 17, 2020 (CN) 202021722504.1

(51) Int. Cl. A61F 5/05

(52) U.S. Cl. CPC A61F 5/05

(57) ABSTRACT

A lightweight neck brace includes a head bracing member, a support member and a rotating mechanism rotatably couple the head bracing member and the support member between an adjustment state and a free state. The rotating mechanism enables the lightweight neck brace to be operable to switch between the adjustment state and the free state. In the adjustment state, the head bracing member can only be adjusted away from the support member, such that a height position of the supported head cannot be lowered. In the free state, the head bracing member can be adjusted away from the support member or close to the support member as desired.

### BACKGROUND OF THE PRESENT INVENTION

### Field of Invention

The invention relates to a neck support device, and more particularly to a rotating mechanism, a lightweight neck brace and an adjustment method thereof.

### Description of Related Arts

The neck acts as a connection between the head and the torso that the heavier head is supported by the neck to the torso. With the function of the neck, the head can flexibly turn left and right or up and down, and at the same time, the neck also bears the pressure generated by the weight of the head. If the head remains in a fixed position for a long time, a greater burden will be brought to the neck. For example, people who work at a desk for a long time often feel neck pain. What's more serious happened while the head is kept in a bad posture for a long time. For example, students may place their heads too close to the book or desk resulting in the occurrence of vision diseases, such as myopia, during the development process thereof, and that their necks also bear undue pressure at the same time. Another example is that since the office workers generally have to keep the same posture for a long time during office hours, their cervical spines bear the full weight of their heads. Under the pressure of the head, in the long run, it is easy to have stiff neck muscles as well as discomfort in the shoulder and neck. Severe cases can cause compression of the vertebral arteries or nerves in the neck that leads to neck and shoulder pain, upper limb pain and other symptoms, which seriously affects the physical and mental health and quality of life.

In addition, the weight of the head itself applies pressure on the cervical spine. On the other hand, during people's daily working and studying life, activities such as tedious work, homework, reading, playing mobile phones, playing games and etc. prolong the bowing time of the people's heads. When the head is bowed downwards, the burden on the cervical spine is further increased. Research results show that when an included angle between the human head and the cervical spine is 0°, the cervical spine bears a weight of 10 lb. to 12 lb. When the human head is bowed by 15°, the cervical spine bears a weight of 27 lb. When the head of the human body is bowed by 30°, the cervical spine bears a weight of 40 lb. When the head of the human body is bowed by 45°, the cervical spine bears a weight of 49 lb. When the head of the human body is bowed by 60°, the cervical spine bears a weight of 60 lb.

The neck brace can keep the neck at an angle and distribute the weight of the head to the trunk. It is because the neck brace for supporting the head is supported on the shoulders, so that the burden applied on the neck can be reduced. However, the conventional neck brace is often designed and used for patients with neck or head illness who need rehabilitation treatment, which leads to a bulky size and volume adapted to support the head on the shoulders while preventing the head to flexibly move and turn. On the other hand, the neck brace itself cannot be adjusted such that the angle of the head and neck remains fixed. Therefore, it is difficult for the user to lower his head or raise his head after wearing the conventional neck brace, that restricting the movement of the head for rehabilitation and health care usage. Apparently, the main target group of users for this type of neck brace is patients with pre-existing illness in the neck or head, which is not suitable to be used for healthy people who like to protect their necks.

### SUMMARY OF THE PRESENT INVENTION

An object of the present invention is to provide a rotating mechanism, a lightweight neck brace and an adjustment method therefor, wherein the lightweight neck brace can be conveniently worn on the user's neck and the relative positions of the user's head and neck can be easily adjusted.

An object of the present invention is to provide a rotating mechanism, a lightweight neck brace and an adjustment method therefor, wherein the lightweight neck brace can support the user's head with a predetermined angle with respect to the neck, so as to maintain a correct posture of the head in relative to the neck of the user by means of the lightweight neck brace.

An object of the present invention is to provide a rotating mechanism, a light-weight neck brace and an adjustment method therefor, wherein the lightweight neck brace can be used to prevent myopia.

An object of the present invention is to provide a rotating mechanism, a lightweight neck brace and an adjustment method therefor, wherein the lightweight neck brace includes a head bracing member and a support member, wherein the head bracing member is supported on the support member while the support member can be supported on the torso to reduce the pressure on the neck caused by the weight of the head.

An object of the present invention is to provide a rotating mechanism, a lightweight neck brace and an adjustment method therefor, wherein an included angle is defined between the head bracing member and the support member of the lightweight neck brace, such that the included angle can be adjusted so as to change a relationship between the head and the neck of the user by adjusting an angle of the head with respect to the neck.

An object of the present invention is to provide a rotating mechanism, a lightweight neck brace, and an adjustment method therefor, wherein after the external force applied for an adjustment of the angle between the head bracing member and the support member, the head bracing member and the support member are retained in a state after the adjustment.

An object of the present invention is to provide a rotating mechanism, a lightweight neck brace, and an adjustment method therefor, wherein the head bracing member of the lightweight neck brace can be supported at a lower jaw and is configured to perform massaging function for providing a massaging effect to the lower jaw.

An object of the present invention is to provide a rotating mechanism, a lightweight neck brace, and an adjustment method therefor, wherein the head bracing member and the support member of the lightweight neck brace are configured to be foldable, and that the head bracing member and the support member are capable of folding up to a smaller size for storage.

An object of the present invention is to provide a rotating mechanism, a lightweight neck brace and an adjustment method therefor, wherein the rotating mechanism is suitable for a head support device, wherein the head support device is configured for wearing on the neck to support the head so as to reduce the compression of the head against the cervical spine of the user, which is beneficial to prevent or alleviate cervical spine diseases.

An object of the present invention is to provide a rotating mechanism, a lightweight neck brace and an adjustment method therefor, wherein the head support device includes a wearing body and a bracing body, the bracing body is rotatably mounted on the wearing body, and that the bracing body is rotatable and switchable between a unidirectional rotation state and a bidirectional rotation state, wherein the bracing body of the head support device merely allows the wearing body to be rotated upwardly during the unidirectional rotation state thereof, and that the bracing body of the head support device only allows the wearing body to be rotated upwardly and downwardly during the bidirectional rotation state thereof, so as to meet the different needs of users and improve the flexibility of the head support device.

An object of the present invention is to provide a rotating mechanism, a lightweight neck brace and an adjustment method therefor, wherein the bracing body of the head support device can only be rotated upward relative to the wearing body to avoid the user's head forces the bracing body to rotate downward relative to the wearing body when the user wants to bow his head, so as to ensure the bracing body of the head support device being always able to support the user's head stably.

An object of the present invention is to provide a rotating mechanism, a lightweight neck brace and an adjustment method therefor, wherein the bracing body of the head support device can be rotated upwardly and downwardly relative to the wearable device, allowing the user to fold up the head support device by rotating the bracing body, so as to reduce the size and volume of the foldable head support device for facilitating the user to store and carry the head support device.

An object of the present invention is to provide a rotating mechanism, a lightweight neck brace and an adjustment method therefor, wherein rotating mechanism is connected with the bracing body and the wearing body so as to switch the bracing body and the wearing body between the unidirectional rotation state and the bidirectional rotation state by means of the rotating mechanism.

According to one aspect of the present invention, the present invention provides a lightweight neck brace, which comprises:
a head bracing member, wherein the head bracing member is adapted to support a head;
a support member, wherein the support member includes a first support arm and a second support arm, wherein the first support arm and the second support arm extend outwards from two ends of the head bracing member respectively to connect with each other so as to be able to form a stable triangular support configuration, wherein the head bracing member is able to be supported on the support member and the support member is adapted to be supported by the trunk; and
two adjustment members, wherein the two adjustment members are respectively provided at two connection positions where the head bracing member rotatably connecting with the first support arm and the second support arm of the support member in such manner that an included angle defined between the head bracing member and the support member is adjustable, wherein the lightweight neck brace has an adjustment state and a free state and is configured to be switchable between the adjustment state and the free state, such that during the adjustment state, the head bracing member is merely adjustable towards a direction away from the support member, so as to prevent a height position of the head being supported from lowering in the adjustment state, and to allow an orientation of the head bracing member being adjusted freely away from the support member or toward the support member in the free state.

According to another aspect of the present invention, the present invention provides a method of adjusting a lightweight neck brace, comprising the following steps:
switching a head bracing member and a support member of the lightweight neck brace between an adjustment state and a free state by means of an outer rotation element and an inner rotation element of each adjustment member of the lightweight neck brace, wherein the head bracing member and the support member of the lightweight neck brace is rotatably connected with each other by means of adjustment members while the head bracing member being supported by the support member is adapted to support the head and the support member is supported by the trunk; allowing the head bracing member to be unidirectionally rotated toward a direction away from the support member and preventing the head bracing member to be rotated towards the support member, during the adjustment state of the lightweight neck brace, when the inner rotation element is engaged with the outer rotation element; and allowing the head bracing member to be freely rotated towards or away from the support member, during the free state of the lightweight neck brace, when the inner rotation element is separated from the outer rotation element.

According to another aspect of the present invention, the present invention provides a rotating mechanism adapted for a head support device, wherein the head support device includes a wearing body and a bracing body movably arranged with the wearing body, wherein the rotating mechanism is configured to connect the wearing body and the bracing body, wherein the rotating mechanism is switchable between a unidirectional rotation state and a bidirectional rotation state, wherein when the rotating mechanism is in the mondirectional rotation state, the rotation mechanism allows the bracing body to merely rotate upward with respect to the wearing body, and when the rotation mechanism is in the bidirectional rotation state, the rotation mechanism allows the bracing body to selectively rotate upwardly and downwardly with respect to the wearing body.

### BRIEF DESCRIPTION OF THE DRAWINGS

FIG. 1 is a perspective view of a lightweight neck brace according to a preferred embodiment of the present invention.

FIG. 2 is an exploded view of the lightweight neck brace according to the above preferred embodiment of the present invention.

FIG. 3 is a schematic view illustrating an application of the lightweight neck brace according to the above preferred embodiment of the present invention.

FIG. 4 is a schematic view illustrating the adjustment of the lightweight neck brace in an adjustment state according to the above preferred embodiment of the present invention.

FIG. 5 is another schematic view of another application of the lightweight neck brace according to the above preferred embodiment of the present invention.

FIG. 6 is a schematic view illustrating the adjustment of the lightweight neck brace in a free state according to the above preferred embodiment of the present invention.

FIG. 7 is a schematic view illustrating an alternative mode of the lightweight neck brace according to the above preferred embodiment of the present invention.

FIG. 8 is a schematic diagram illustrating another alternative mode of the lightweight neck brace according to the above preferred embodiment of the present invention.

FIG. 9 is a perspective view of a head support device during a first state according to the above preferred embodiment of the present invention.

FIG. 10 is a perspective view of the head support device during a second state according to the above preferred embodiment of the present invention.

FIG. 11 is an exploded view of the head support device according to the above preferred embodiment of the present invention.

FIG. 12A is a partial sectional view of a partial structure of the head support device according to the above preferred embodiment of the present invention.

FIG. 12B is a partial sectional view of a partial structure of the head support device according to the above preferred embodiment of the present invention.

FIG. 13 is a schematic view showing an application of the head support device according to the above preferred embodiment of the present invention.

FIG. 14 is a schematic diagram showing the application of the head support device according to the above preferred embodiment of the present invention, wherein a process of upward adjustment of the head support device is illustrated.

FIG. 15 is a schematic view showing another application of the head support device according to the above preferred embodiment of the present invention.

FIG. 16 is a schematic view showing the another application of the head support device according to the above preferred embodiment of the present invention, wherein a process of downward adjustment of the head support device is illustrated.

FIG. 17 is a schematic view showing another application of the head support device according to the above preferred embodiment of the present invention.

FIG. 18 is a schematic diagram showing the another application of the head support device according to the above preferred embodiment of the present invention, wherein a process of downward adjustment of the head support device is illustrated.

### DETAILED DESCRIPTION OF THE PREFERRED EMBODIMENT

The following description is to disclose the invention to enable those skilled in the art to practice the invention. The preferred embodiments described below are given by way of example only, and other obvious modifications is apparent to those skilled in the art. The basic principles of the invention defined in the following description may be applied to other embodiments, variations, improvements, equivalents, and other technical solutions without departing from the spirit and scope of the invention.

It should be understood by those skilled in the art that in the disclosure of the present invention, the orientation or positional relationship indicated by the terms “longitudinal”, “transversal”, “upper”, “lower”, “front”, “rear”, “left”, “right”, “vertical”, “horizontal”, “top”, “bottom”, “inner”, “outer”, and etc. is based on the orientation or positional relationship shown in the drawings, which is only for the convenience of describing the present invention and to simplify the description, rather than to indicate or imply that the device or element referred to must have a particular orientation, be constructed and operated in a particular orientation, and thus the above terms should not be construed as limiting the invention.

It should be understood that the term “a” should be understood as “at least one” or “one or more”, that is, in one embodiment, the number of an element may be one, while in another embodiment, the number of the element may be plural, and the term “one” should not be understood as a limitation on the number.

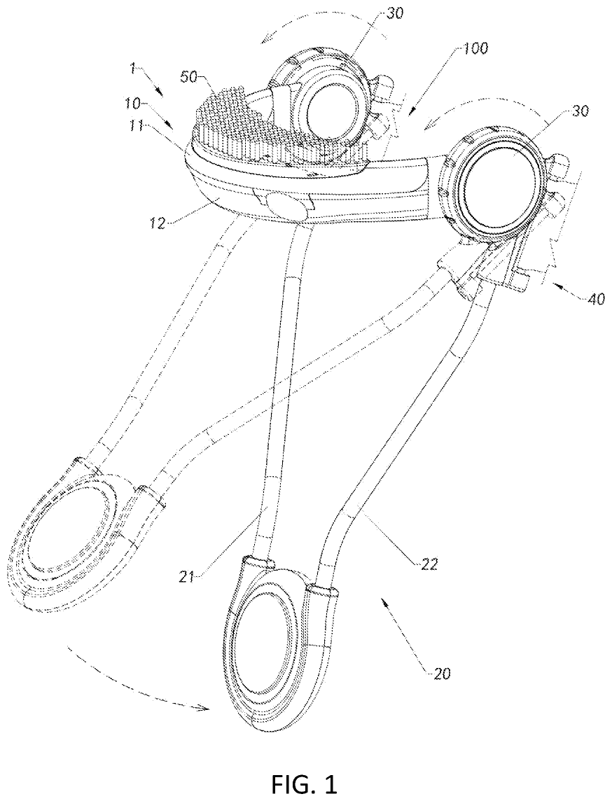

Referring to FIG. 1 to FIG. 6, a lightweight neck brace 1 according to a preferred embodiment of the present invention is illustrated, wherein the lightweight neck brace 1 is configured to support the user's head on the torso to reduce the pressure applied by the weight of the head to the neck, and that the lightweight neck brace 1 can be designed to be lighter and adjustable to meet the needs of users. In comparison with the conventional one-piece clumsy neck brace, the lightweight neck brace 1 of the present invention is very convenient to use.

In particular, the lightweight neck brace 1 includes a head bracing member 10 and a support member 20, wherein the head bracing member 10 is configured to support the head of a user in a desired position, and the support member 10 is configured to be supported by the support member 20 which is capable of being supported on the torso of the user. For example, it can be supported on the chest, shoulders and/or abdomen of the user.

It is worth mentioning that the lightweight neck brace 1 can be used not only to humans, but also to animals, as long as the head bracing member 10 and the support member 20 are adaptively adjusted, thereby modifications are still within the scope of the present invention.

The lightweight neck brace 1 forms and defines a wearing channel 100, such that the user's neck can be positioned within the wearing channel 100 so as to wear the lightweight neck brace 1 in position. A size of the wearing channel 100 is arranged to be adjustable to fit different users. Optionally, the user's head may be positioned in the wearing channel 100 in order to wear the lightweight neck brace 1.

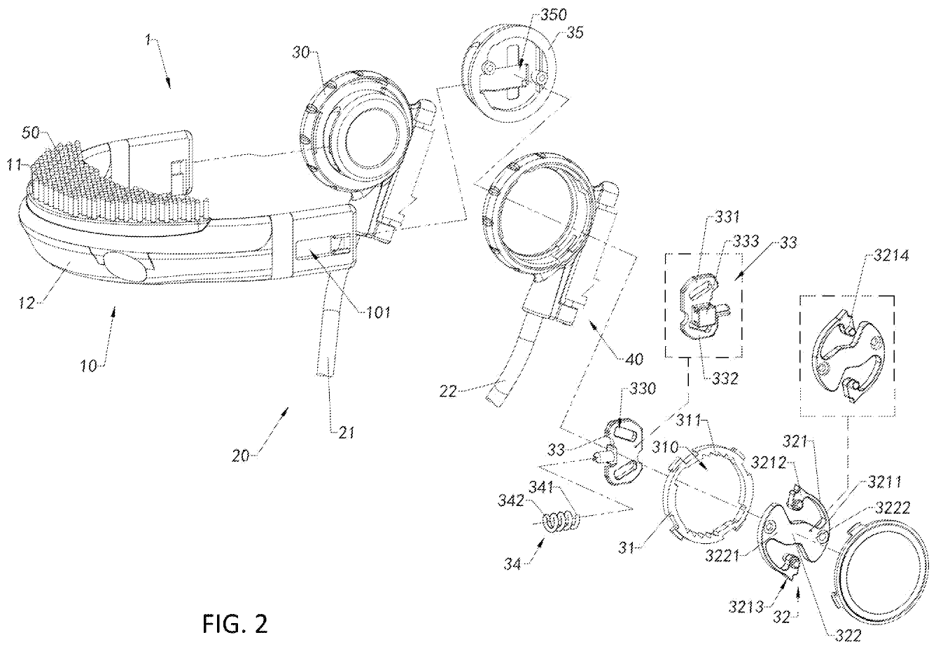

Referring to FIG. 1 and FIG. 2, the support member 20 includes a first support arm 21 and a second support arm 22, wherein the first support arm 21 and the second support arm 22 are respectively extended outwards from two ends of the head bracing member 10 until being connected with each other at one end thereof. Specifically, one end of the first support arm 21 and one end of the second support arm 22 are rotatably connected to two ends of the head bracing member 10 respectively while another end of the first support arm 21 is connected with another rend of the second support arm 22, such that the first support arm 21 and the second support arm 22 of the support member 20 and the head bracing member 10 construct a stable triangular configuration for stably supporting the head with respect to the torso of the user.

The support member 20 and the head bracing member 10 are arranged to define an included angle therebetween, wherein a size of the included angle is preferably arranged to be adjustable. In particular, the lightweight neck brace 1 further includes at least one adjustment member 30, which is arranged at a connection position between the support member 20 and the head bracing member 10, such that the included angle defined between the support member 20 and the head bracing member 10 is able to be adjusted by means of the at least one adjustment member 30, and that relative positions of the head bracing member 10 and the support member 20 after adjustment are able to be maintained. Preferably, in the preferred embodiment of the lightweight neck brace 1 as shown in FIG. 1 to FIG. 6, the lightweight neck brace 1 includes two adjustment members 30, one of the two adjustment members 30 is arranged at where the first support arm 21 connecting with the head bracing member 10, and another one of the two adjustment members 30 is provided at where the second support arm 22 connecting with the head bracing member 10.

The head bracing member 10 can be pivotally folded towards the support member 20, such that the lightweight neck brace 1 can be folded into a smaller size, thereby making the lightweight neck brace 1 easy to carry. The head bracing member 10 is able to be supported at a predetermined angle with respect to the support member 20, so that the head bracing member 10 is capable of supporting the head at a desired angle.

Further, the lightweight neck brace 1 has a function for preventing myopia. When a user sits at a desk or in front of a computer, the user's head tends to approach the desk or the computer. In the long run, the user may become short-sighted. The head bracing member 10 and the support member 20 of the lightweight neck brace 1 can be adjusted to have a desired angle therebetween and be maintained at the desired angle, so as to prevent the head from approaching the table or the computer, thereby providing the function for preventing myopia.

Furthermore, the head bracing member 10 and the support member 20 of the lightweight neck brace 1 are arranged to be unidirectionally adjustable that, when the lightweight neck brace 1 is worn on the user's body, the head bracing member 10 is unable to be adjusted toward the support member 20 and thus the head cannot be bowed further lower that is conducive to protecting the user's vision, and that the head bracing member 10 is able to adjusted in a direction away from the support member 20, so as to adapt for different postures and usage requirements of users.

It is worth noticing that another advantage for the head bracing member 10 and the support 20 being arranged for unidirectional adjustment is that, when the head is supported by the head bracing member 10 and has a tendency of moving downward, since the head bracing member 10 is not allowed to move toward the support member 20 and is restricted to merely move in a direction away from the support member 20, thereby reducing the possibility of the user's head moving downward gradually unconsciously due to the insufficient connection strength between the head bracing member 10 and the support member 20.

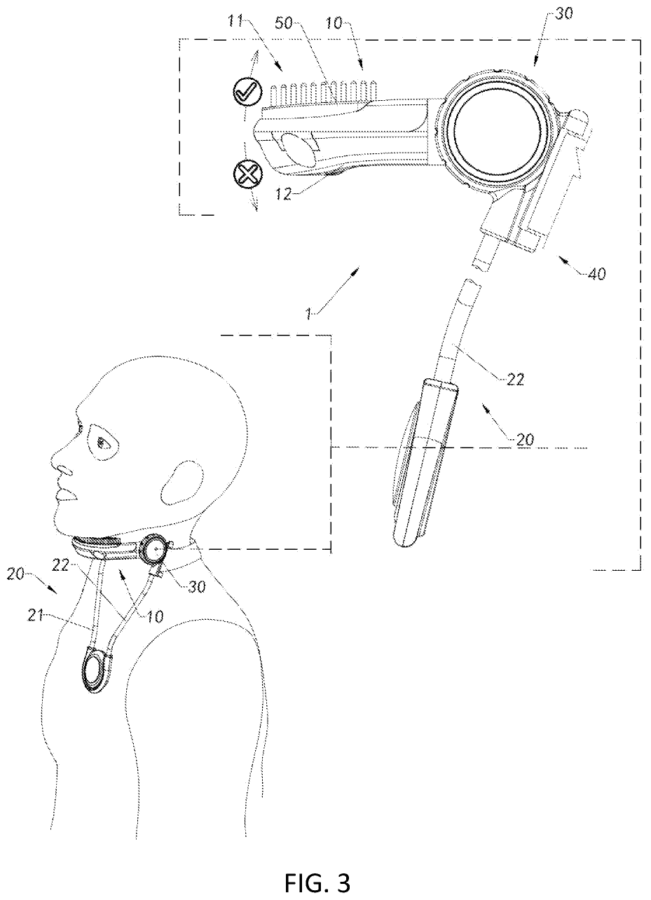

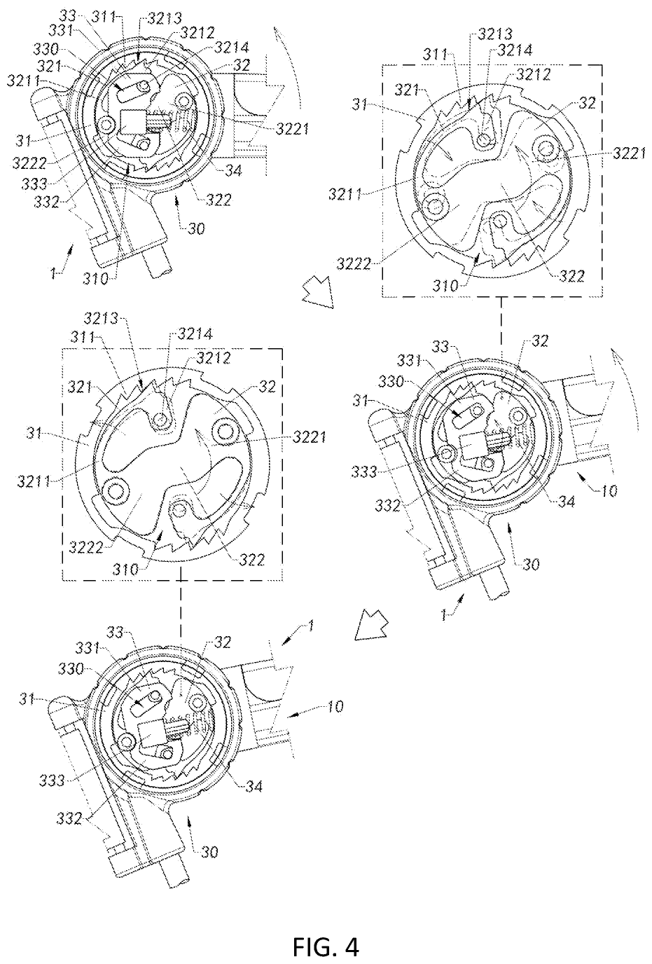

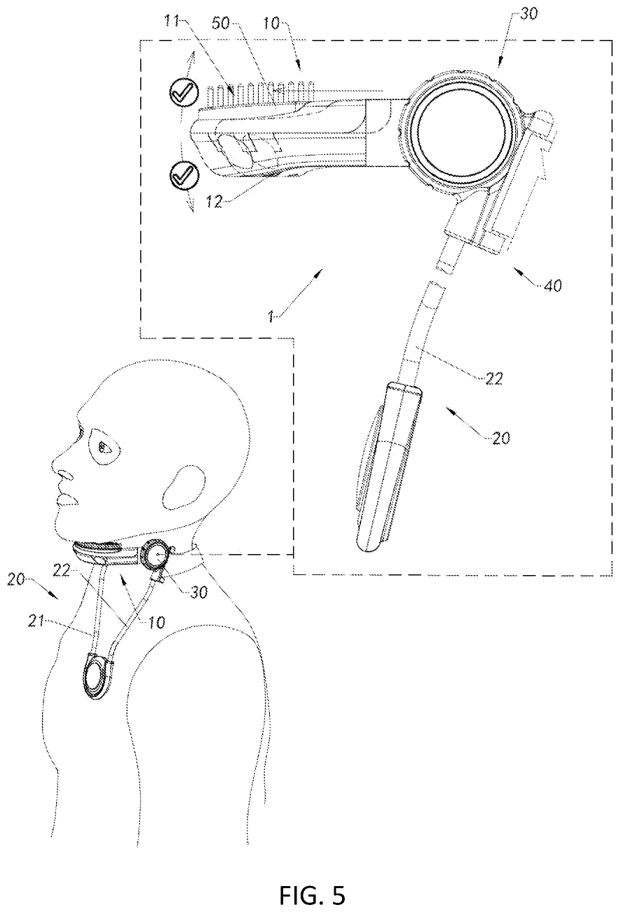

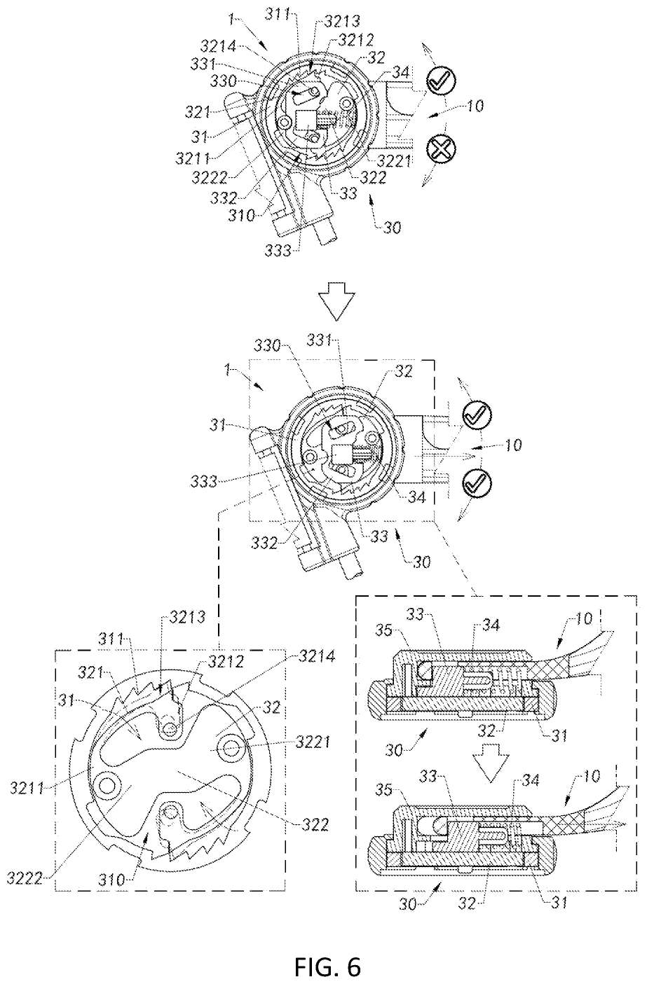

In view of the lightweight neck brace 1, the lightweight neck brace 1 has an adjustment state and a free state. In the adjustment state, referring to FIG. 3 and FIG. 4, the head bracing member 10 of the lightweight neck brace 1 can only preform unidirectional adjustment with respect to the support member 20 and is able to be fixed in the state after adjustment that the head bracing member 10 is unable to move toward the support member 20 so as to stably support the head by the head bracing member 10. In the free state, referring to FIG. 5 and FIG. 6, the head bracing member 10 of the lightweight neck brace 1 can be adjusted toward the support member 20 and can also be adjusted toward a direction away from the support member 20. In other words, a rotation direction of the head bracing member 10 is not restricted. The head bracing member 10 can be adjusted to be folded over the support member 20. It can be understood that the arrangement of the head bracing member 10 and the support member 20 in unidirectional adjustment means that, when the lightweight neck support 1 is in the adjustment state, the head bracing member 10 of the lightweight neck support 1 is only allowed to be adjusted in one direction with respect to the support member 20. In other words, when the lightweight neck brace 1 is in the free state, the adjustment direction of the head bracing member 10 with respect to the support member 20 is not restricted.

By adjusting the adjustment member 30, the lightweight neck brace 1 can be switched between the adjustment state and the free state. In particular, when no external force is applied to the at least one adjustment member 30, the lightweight neck brace 1 is in the adjustment state, and correspondingly, when an external force is applied to the at least one adjustment member 30, the lightweight neck brace 1 can be switched from the adjustment state to the free state.

In detail, referring to FIG. 2, each adjustment member 30 includes an outer rotation element 31 and an inner rotation element 32, wherein the outer rotation element 31 is arranged at the support member 20 and the inner rotation element 32 is arranged at the head bracing member 10. The inner rotating member 32 is rotatably connected with the outer rotating member 31. Through the rotation of the outer rotating member 31 and the inner rotating member 32, the size of the included angle defined between the support member 20 and the head bracing member 10 is adjusted correspondingly. Alternatively, the outer rotating member 31 can also be arranged at the head bracing member 10 while the inner rotating member 32 is arranged at the supporting portion 20.

Referring to FIG. 2, the head bracing member 10 has two corresponding end portions, wherein two inner rotating members 32 of two adjustment members 30 are respectively provided at the two end portions of the head bracing member 10 and the two outer rotation elements 31 of the two adjustment members 30 are provided at an upper end portion of the first support arm 21 and an upper end portion of the second support arm 22 respectively, wherein the inner rotating members 31 are rotatably coupled with the outer rotating members 31 respectively, such that the two end portions of the head bracing member 10 are arranged with the upper end portions of the first support arm 21 and the second support arm 22 through the two adjustment members 30 respectively.

Further, a receiving cavity 310 is defined in each of the outer rotating members 31, wherein the inner rotating members 32 are respectively accommodated in the receiving cavities 310. Each outer rotating member 31 has an inner wall, wherein at least a portion of the inner wall is recessed to form at least two sets of unidirectional tooth grooves 311 which are configured for the respective inner rotating member 32 to be rotated to engage with the unidirectional tooth grooves 311 of the outer rotation element 31. When the outer rotating member 31 or the inner rotating member 32 is applied with a force, the inner rotating member 32 is configured to be able to disengage with one of the unidirectional tooth grooves and then engage with the next unidirectional tooth groove thereof.

Specifically, when the lightweight neck brace 1 is in the adjustment state, the inner rotating member 32 is originally engaged with one tooth groove of each set of the unidirectional tooth grooves 311 of the outer rotating member 31 so that the head bracing member 10 and the support member 20 remain engaged and fixed with each other. Applying a force to the inner rotating member 32 or the outer rotating member 31 would disengage the inner rotating member 32 from the one tooth groove of each set of the unidirectional tooth grooves 311, and then engage in the next tooth groove of each set of the unidirectional tooth grooves so that the head bracing member 10 returns to a fixed state with respect to the support member 20 while a position of the head bracing member 10 with respect to the support 20 has been adjusted and the included angle between the head bracing member 10 and the support member 20 is enlarged.

The outer rotating member 31 may be implemented as a toothed ring body, and each set of the unidirectional tooth grooves 311 is formed in the inner wall of the outer rotating member 31. Two or more sets of the unidirectional tooth grooves 311 are provided and implemented in symmetrical manner.

The inner rotating member 32 is provided with at least one connecting arm 321 which is configured to be engaged with each unidirectional tooth groove 311 of the set of unidirectional tooth grooves 311 of the outer rotating member 31. Two or more connecting arms 321 may be implemented in symmetrical manner. A predetermine interval is retained between two adjacent connecting arms 32. According to the preferred embodiment, two connecting arms 321 are implemented centrally symmetrical such that the two connecting arms 321 are able to be engaged with the two sets of the unidirectional tooth grooves 311 of the outer rotating member 31 respectively.

Specifically, each inner rotating member 32 includes a base body 322 and the two connecting arms 321 are respectively extended from two sides of the base body 322 symmetrically. The base body 322 is fixed to the head bracing member 10. Each connecting arm 321 has a connecting end 3211 and a free end 3212 extended from the connecting end 3211, wherein the connecting end 3211 of the connecting arm 321 is integrally connected to the base body 322 and the free end 3212 of the connecting arm 321 is configured to engage with the tooth grooves 311 of the outer rotating member 31. Preferably, the free end 3211 of the connecting arm 321 integrally extends outwardly from the base body 322 in a suspending manner, allowing the connecting arm 321 to be forced to deform towards the base body 322 under a pressure and return to an initial state thereof when the pressure is released.

The connecting arm 321 has a supporting surface 3213 formed thereon. The supporting surface 3213 can be formed at the free end 3212 of the connecting arm 321, wherein the supporting surface 3213 can be pressed against the respective unidirectional tooth groove 311 of the outer rotating member 31. The support surface 3213 can be an inclining surface that is capable of matching with an inclined surface of each of the tooth grooves 311 of the outer rotating member 31 correspondingly.

The inner rotating member 32 is installed in the outer rotating member 31 of each adjustment member 30, such that the connecting arms 321 are compressed and pressed against the inner wall of the outer rotating member 31. When the inner rotating member 32 and the outer rotating member 31 rotate relative to each other, the inner rotating member 32 is able to be rotated until the supporting surface 3213 of each connecting arms 321 thereof being engaged with one of the unidirectional tooth grooves 311 of the outer rotating member 31 respectively. When the inner rotating member 32 and the outer rotating member 31 are further rotated relative to each other, the inner rotating member 32 is able to be rotated to allow the support surface 3213 of each connecting arms 321 to be engaged with the next one of the unidirectional tooth grooves 311 of the outer rotating member 31.

The extending direction of the connecting arm 321 is arranged along the inner wall of the outer rotating member 31, or that the extending direction of the connecting arm 321 is arranged along a rotating direction of the inner rotating member 32 and the outer rotating member 31. When the outer rotating member 31 and the inner rotating member 32 rotate relative to each other, since the extending direction of the connecting arm 321 is arranged along the rotating direction of the outer rotating member 31, the inner rotating member 32 and the force applied on the connecting arm 321 is mainly caused by an extrusion effect on the free end 3212 of the connecting arm 321, and thus the reaction force formed at where the connecting arm 321 connecting with the base body 322 is small, that facilitates a reduction of the worn out of the connecting portion of the connecting arm 321 of the inner rotating member 32 and the base body 322, and also facilitates the rotation of the outer rotating member 31 and the inner rotating member 32 while reducing the resistance therebetween.

The user is capable of rotating the outer rotating member 31 and the inner rotating member 32 with less force. When the inner rotating member 32 is rotated until the supporting surfaces 3213 of the connecting arms 321 is aligned with the respective unidirectional tooth grooves 311 of the outer rotating member 32, since the head bracing member 10 and the support member 20 cannot rotate in an opposite direction, the head bracing member 10 is able to be stably retained in the adjusted position.

In other words, the outer rotating member 31 is mounted to the support member 20 and the inner rotating member 32 is mounted to the head bracing member 10, such that the outer rotating member 31 and the inner rotating member 32 are capable of rotating with respect to each other, thereby the head bracing member 10 is merely allowed to be rotated in a direction away from the support member 20 while the lightweight neck support 1 is in the adjustment state.

Since the supporting surfaces 3213 of the connecting arms 321 of the inner rotating member 32 are pressed against the sets of unidirectional tooth grooves 311 of the outer rotating member 31 respectively, the outer rotating member 31 and the inner rotating member 32 cannot freely rotate clockwise or counterclockwise that the inner rotating member 32 can only rotate with respect to the outer rotating member 31 along the direction of the unidirectional tooth grooves 311. When the inner rotating member 32 rotates along the direction of the unidirectional tooth grooves 311, the head bracing member 10 pivotally rotates in a direction away from the support member 20. When the inner rotating member 32 is rotated until the supporting surfaces 3213 of the connecting arms 321 of the inner rotating member 32 are engaged with the sets of unidirectional tooth grooves 311 of the outer rotating member 31 respectively, the head bracing member 10 is retained with the support member 20 that the head bracing member 10 is unable to rotate in the direction toward the support member 20.

Referring to FIG. 2, the base body 322 has a first end portion 3221 and a second end portion 3222 opposing to the first end portion 3221, wherein one of the connecting arms 321 is connected to the first end portion 3221 and suspendingly extends toward the second end portion 3222, and another one of the connecting arms 321 is connected to the second end portion 3222 and suspendingly extends toward the first end portion 3221. In other words, the free end 3212 of the connecting arm 321 connected to the first end portion 3221 maintains a predetermined distance with the second end portion 3222 that allows the connecting arm 321 to deform toward the base body 322 under a pressing force applied thereto, and that the free end 3212 of the connecting arm 321 connected to the second end portion 3221 maintains a predetermined distance with the first end portion 3221 that allows the connecting arm 321 to deform toward the base body 322 under a pressing force applied thereto.

The distance from the free ends 3212 of the connecting arms 321 to the base body 322 is variable within a predetermined range. For example, when the supporting surfaces 3213 of the connecting arms 321 are pressed against the sets of unidirectional tooth grooves 311 formed in the inner wall of the outer rotating member 31 and disengaging from the current unidirectional tooth grooves 311 respectively to reengage with the next unidirectional tooth grooves 311 respectively, the distance between the free ends 3212 of the connecting arms 321 and the base body 322 changes correspondingly during the disengaging and reengaging process.

When the outer rotating member 31 rotates relative to the inner rotating member 32, the force exerted by the outer rotating member 31 is capable of pressing the free ends 3212 of the connecting arms 321 inwardly where each of the connecting arms 321 is a resilient element having a predetermined elasticity to maintain its contact with the outer rotating member 31. Since the connecting arm 321 is arc-shaped and extends along a rotation direction of the outer rotating member 31 and the inner rotating member 32, the pressing force of each connecting arm 321 is mainly applied to the free end 3212 thereof, so that the force acting on the connecting end 3211 of each connecting arm 321 is reduced. In view of the connecting arms 321 of the inner rotating member 32, the connecting ends 3211 of the connecting arms 321 will not cause too much resistance during the rotation of the outer rotating member 31 and the inner rotating member 32, thereby facilitating the rotation of the outer rotating member 31 and the inner rotating member 32 by the user.

Furthermore, for the uniformity of the force applied on the outer rotating member 31 and the inner rotating member 32 and the smoothness of the rotation of the outer rotating member 31 and the inner rotating member 32, the two connecting arms 321 are arranged coaxially and symmetrically, so that when the outer rotating member 31 rotates relative to the inner rotating member 32, the forces applied on the free ends 3212 of the two connecting arms 321 are along the same rotation direction.

The lightweight neck brace 1 is controllably switchable between the adjustment state and the free state. The adjustment member 30 further comprises an adjusting element 33, wherein the adjusting element 33 is operable to allow the lightweight neck brace 1 to be switched between the adjustment state and the free state.

In the adjustment state, the free ends 3212 of the connecting arms 321 of the inner rotating member 32 are restricted by the adjusting element 33, so that the inner rotating member 32 is retained to pressing against the outer rotating member 31, and thus the inner rotating member 32 and the outer rotating member 31 are unable to rotate freely.

In the free state, the adjusting element 33 releases the free end 3212 of the connecting arm 321 of the inner rotating member 32, so that the inner rotating member 32 can be separated from the outer rotating member 31, and thus the inner rotating member 32 and the outer rotating member 31 are able to rotate in either clockwise or counterclockwise direction.

Specifically, the adjusting element 33 has at least one restriction slot 330 such that the connecting arm 321 of the inner rotating member 32 is able to be restricted in the restriction slot 330 to prevent separating from the outer rotating member 31. In the preferred embodiment, since two connecting arms 321 are provided, there are two restriction slots 330 provided correspondingly.

Each connecting arm 321 may be embodied to have a restricting pin 3214 protruded outwardly and extended in an axial direction thereof. The restricting pin 3214 of the connecting arm 321 is configured to be restricted in the restriction slot 330 of the adjusting element 33.

The restricting pins 3214 of the two connecting arms 321 may be arranged symmetrically, for example, arranged coaxially and symmetrically arranged, that is the two restriction slots 330 of the adjusting element 33 may also be arranged coaxially and symmetrically. Correspondingly, the two connecting arms 321 may be arranged coaxially and symmetrically.

When the adjusting element 33 is adjusted to drive the free ends 3212 of the connecting arms 321 to move inwardly to disengage the unidirectional tooth grooves 311 of the outer rotating member 31, the restriction pins 3214 restricted in the two restriction slots 330 are driven by the adjusting element 33 to move synchronously, so that the two connecting arms 321 are synchronously disengaged with the outer rotating member 31 and, at this time, the lightweight neck brace 1 is adjusted to the free state.

More specifically, each adjusting element 33 includes a first restricting wing 331 and a second restricting wing 332, wherein the two restriction slots 330 are formed in the first restricting wing 331 and the second restricting wing 332 respectively. The adjusting element 33 further includes a mounting seat 333, wherein the first restricting wing 331 and the second restricting wing 332 are respectively extended from two edges of the mounting seat 333. The two mounting seats 333 are respectively installed at the two ends portions of the head bracing member 10.

The first restricting wing 331 is integrally connected with the second restricting wing 332 and the mounting seat 333 is provided on one side of the first restricting wing 331 and the second restricting wing 332. The user can operate the head bracing member 10, such as pulling the head bracing member 10 outwards, to drive the adjusting element 33 to move so as to release the engagement of the supporting surfaces 3213 of the connecting arms 321 of the inner rotating member 32 from the unidirectional tooth grooves 311 of the outer rotating member 31.

Each restriction slot 330 is arranged in a slant manner. When the adjusting element 33 is driven by the head bracing member 10 to move, the adjusting member 33 moves with the restriction slot 330 thereof to restrict the restricting pin 3214 to move inwardly while allowing the free end 3212 of the connecting arm 321 to move inwardly until the supporting surface 3213 of the connecting arm 321 disengaging from the unidirectional tooth groove 311 of the outer rotating member 31.

The extending direction of the restriction slots 330 as well as the moving direction of the adjusting element 33 may be slanting. Each adjusting element 33 has a first position and a second position. When the adjusting element 33 is located in the first position, the connecting arms 321 of the inner rotating member 32 is restricted by the adjusting element 33 to retain pressing against the outer rotating member 31. When the adjusting element 33 is located in the second position, the connecting arms 321 of the inner rotating member 32 are compressed in a direction away form the outer rotating member 31 to release the inner rotating member 32. When the adjusting element 33 is moved from the first position to the second position, the slanted restriction slot 330 retains the restriction of the connecting arm 321 and pulls the free end 3212 of the connecting arm 321 away from the unidirectional tooth grooves 311 of the outer rotating member 31.

It is understood that the adjusting element 33 is arranged to be restricted that the adjusting element 33 is able to move from the first position to the second position, or from the second position to the first position but is not allowed to extend and move in other directions.

More specifically, when the restricting pins 3214 of the connecting arms 321 of the inner rotating member 32 are respectively located at the same ends of the two restriction slots 330, a first distance is defined between the two restricting pins 3214. When the restricting pins 3214 of the connecting arms 321 of the inner rotating member 32 are respectively restricted at the other ends of the two restriction slots 330, a second distance is defined between the two restricting pins 3214. The first distance is greater than the second distance, so that when the restricting pins 3214 are respectively restricted to the two ends of the restriction slot 330, the relative positions of the two restricting pins 3214 are changed that changes the positions of the connecting arms 321, thereby the lightweight neck brace 1 can be switched between the free state and the adjustment state.

When the two restricting pins 3214 are moved closer to each other, the two connecting arms 321 are moved toward the base body 322 and away from the unidirectional tooth grooves 311 of the outer rotating member 31, so that the supporting surface 3213 is released from engaging with the unidirectional tooth groove 311 and the lightweight neck brace 1 is in the free state.

When the two restricting pins 3214 are moved away from each other, the two connecting arms 321 are moved toward the outer rotating member 31, so that the supporting surface 3213 moves toward the unidirectional tooth grooves 311 of the outer rotating member 31 until pressing against one of the unidirectional tooth grooves 311, thereby the lightweight neck brace 1 is in the adjustment state.

The adjusting element 33 is positioned at one side of the inner rotating member 32. Each restricting pin 3214 is positioned in an axial direction. The state of the lightweight neck brace 1 can be changed by changing the relative positions of the adjusting element 33 and the inner rotating member 32.

Furthermore, the two restriction slots 330 are arranged in “/\” shape, that is the distance between the two restriction slots 330 is gradually enlarged from one end to another end thereof.

Furthermore, the adjustment member 30 may further include a resilient connecting element 34, wherein the adjusting element 33 installed to the head bracing member 10 is elastically supported by the resilient connecting element 34.

The resilient connecting element 34 is positioned in a movable direction of the adjusting element 33. When the adjusting element 33 moves from the first position to the second position, the resilient connecting element 34 is further compressed. When the adjusting element 33 moves from the second position to the first position, the pressing force previously applied to the resilient connecting element 34 is weakened.

By means of the resilient connecting element 34, the adjusting element 33 can be elastically supported at the first position. When the user exerts a force on the head bracing member 10 to pull the head bracing member 10 outward, the adjusting element 33 can be adjusted to the second position. When the user releases the exerting force, under the effect of the resilient connecting element 34, the adjusting element 33 can automatically return to the first position.

More specifically, each adjustment member 30 further includes an adjustment member installation casing 35, wherein the adjustment member installation casing 35 forms an installation cavity 350 therein, wherein the adjusting element 33 is accommodated in the installation cavity 350 of the adjustment member installation casing 35. The two end portions of the head bracing member 10 are movably inserted and mounted to the two adjustment member installation casings 35 respectively. The base bodies 322 of the inner rotating members 32 are mounted in the adjustment member installation casings 35 respectively, so as to fixedly connect the two adjustment member installation casings 35 to the two end portions of the head bracing member 10 respectively. The adjusting element 33 is positioned between the inner rotating member 32 and the adjustment member mounting housing 35.

The adjusting element 33 is installed in the adjustment member attachment housing 35 and mounted to the head bracing member 10. The adjusting element 33 can be driven by the head bracing member 10 to move synchronously.

The resilient connecting element 34 has a first connecting end 341 and a second connecting end 342 corresponding to the first connecting end 341, wherein the first connecting end 341 of the resilient connecting element 34 is abutted against the mounting seat 333 of the adjusting element 33 or connected to the mounting seat 333 of the adjusting element 33. The second connecting end 342 of the resilient connecting element 34 is arranged to be abutted against the adjustment member installation casing 35 mounted to the head bracing member 10. In other words, the resilient connecting element 34 is positioned between the adjustment member installation casing 35 and the adjusting element 33 of the head bracing member 10. Since the head bracing member 10 can drive the resilient connecting element 34 to move together synchronically, for example, during the outwardly moving of the head bracing member 10, a distance between the adjusting element 33 and the adjustment member installation casing 35 of the head bracing member 10 is shortened, and thus the resilient connecting element 34 is compressed. Thereby, when the head bracing member 10 does not move outwards with the adjusting element 33, the compression of resilient connecting element 34 is released and the resilient connecting element 34 stretches and resets to the original position thereof.

It is worth noting that the reset adjusting element 33 can then drive the head bracing member 10 to reset.

It can be understood that, in the preferred embodiment, the inner rotating member 32 is installed to the head bracing member 10 for description, and the adjusting element 33 is configured to be linked with the head bracing member 10. According to an alternative mode of the preferred embodiment of the present invention, the inner rotating member 32 may also be installed to the support member 20 and the adjusting element 33 may also be configured to be linked with the support member 20, so that it can be switching between the adjustment state and the free state by pulling the support member 20.

In view of the adjusting element 33, each end portion of the head bracing member 10 has a guiding slot 101 provided therein, such that the mounting seat 333 of the adjusting element 33 is configured to be installed in the guiding slot 101 so as to be guided to move forth and back along the guiding slot 101. The guiding slot 101 is configured to guide the moving direction of the adjusting element 33 so as to ensure the inner rotating member 32 can be released when the adjusting element 33 moves outwardly to the second position. When the adjusting element 33 moves inwardly to the first position, the inner rotating member 32 can be pressed against the outer rotating member 31 again.

It is worth noting that, according to the preferred embodiment of the present invention, the adjusting element 33 is arranged to be electrically driven, such that after an operation of the user, the adjusting element 33 can automatically switch between the first position and the second position. For example, the user can press a button to switch the lightweight neck brace 1 between the adjustment state and the free state.

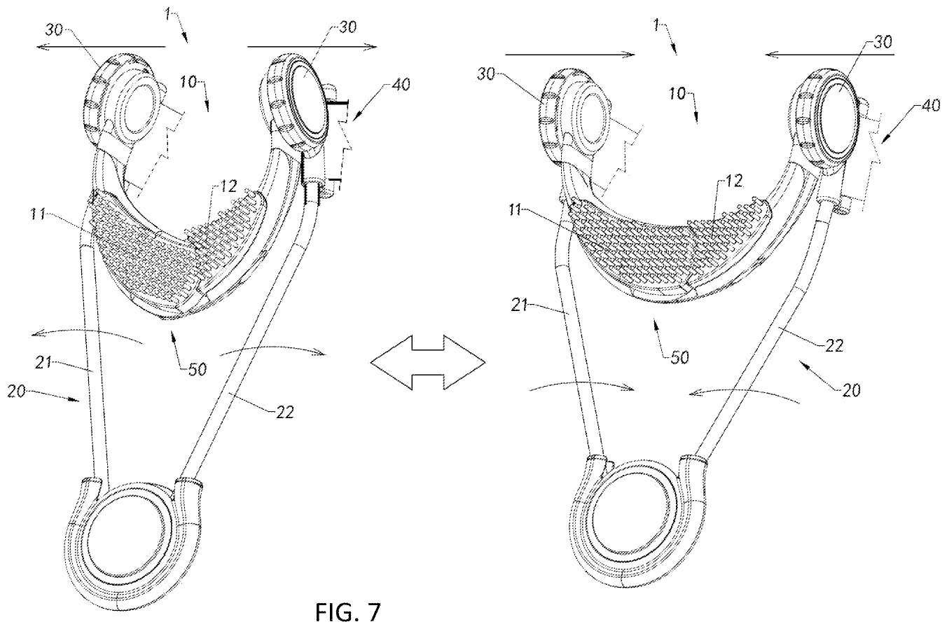

Further, the head bracing member 10 includes a first bracing arm 11 and a second bracing arm 12, wherein the first bracing arm 11 is rotatably connected to the first support arm 21 of the support member 20 through one of the adjustment members 30, and that the second bracing arm 12 is rotatably connected to the second support arm of the support member 20 through another one of the adjustment members 30. The first bracing arm 11 is pivotally connected to the second bracing arm 12. The first support arm 21 is pivotally connected to the second support arm 22. The folding of the first bracing arm 11 towards the second bracing arm 12 is not shown in FIG. 1 to FIG. 6 and please refers to FIG. 7.

When the head bracing member 10 is pivotally folded towards the support member 20, the first bracing arm 11 can be folded towards the second bracing arm 12, and the first support arm 21 can be folded towards the second support arm 22, so as to fold the entire lightweight neck brace 1 to a smaller size.

It is worth noting that an included angle is defined between the first support arm 21 and the second support arm 22 and such included angle can be retained at the position after adjustment. An included angle is defined between the first bracing arm 11 and the second support arm 11 and such included angle can be retained at the position after adjustment. Thereby, the light neck support 1 is capable of fitting different sizes of various users, such as users with different head sizes.

Further, referring to FIG. 1 to FIG. 6, the lightweight neck brace 1 includes a mounting member 40, wherein the mounting member 40 is mounted to the head bracing member 10 to assist the head bracing member 10 to be held in position. In the preferred embodiment, the head bracing member 10 is supported at a lower jaw position of the head, and that the head bracing member 10 does not completely surround the head so as to leave some space for facilitating the user to wear. The mounting member 40 is mounted to the head bracing member 10, and that a distance is defined between the two end portions of the head bracing member 10 and the mounting member 40 is extended between the two end portions of the head bracing member 10, so that the head bracing member 10 can be better worn on the user for head bracing. The head bracing member 10 and the mounting member 40 jointly forms a wearing channel 100 which is adjustable in size, so that the lightweight neck brace 1 can be adaptable to different head sizes of the users.

The mounting member 40 may be, but not limited to, implemented as a strip, a strap with loop and hook fasteners, a string or etc.,

The mounting member 40 can be received in the adjustment member 30 that, when the mounting member 40 is needed to be used, the mounting member 40 can be pulled out from the adjustment member 30.

Further, the lightweight neck brace 1 includes a function portion 50, wherein the function portion 50 is arranged on the head bracing member 10. When the head bracing member 10 supports a lower jaw of the user, the function portion 50 is adapted to fit the lower jaw of the user and to provide a supporting effect to the lower jaw of the user. The function portion 50 can provide a massage effect to the lower jaw, wherein the function portion 50 is provided with protrusions or granular particles adapted for providing massage effect. The function portion 50 may also be configured to provide vibrating function and its relative electrical components can be installed inside the head bracing member 10.

The functional portion 50 may be configured to have resilient ability so as to allow a fine adjustment with respect to the position of the user's head. For example, when the weight of the user's head is relatively heavier, the function portion 50 undergoes a relatively larger deformation, and that when the weight of the user's head is relatively lighter, the function portion 50 undergoes a relatively smaller deformation. The function portion 50 may be implemented in the form of plastic, sponge, cloth bag filled with particles, or the like.

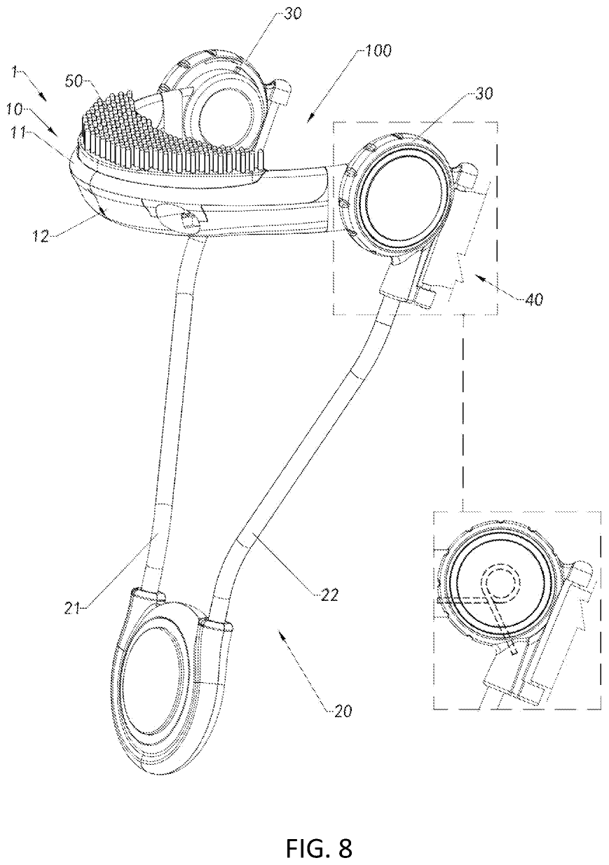

Referring to FIG. 8, an alternative mode of the preferred embodiment of the lightweight neck brace 1 according to the present invention is illustrated. The difference between this alternative mode and the preferred embodiment illustrated in FIG. 1 to FIG. 6 is mainly the adjustment member 30, which is implemented as a torsion spring configuration arranged between the head bracing member 10 and the support member 20.

The head bracing member 10 is supported on the support member 20 through the adjustment member 30. Since a torsion spring has two outstretched extending arms and, under the effect of its own elasticity, an included angle between the two extending arms is unable to be continuously decreased beyond a predetermined angular range. the user is wearing the lightweight neck brace 1, by exerting a force to the adjustment member 30 through the head bracing member 10 and the support member 20 respectively, such as the weight of the user's head, the included angle between the two extending arms of the torsion spring reduces until the angle between two extending arms of the torsion spring is reduced to a predetermined range that the user is unable to continuously move the head downwards. In other words, the user cannot lower his or her head.

When the user wants to adjust the lightweight neck brace 1, the user can only adjust the lightweight neck brace 1 upwards to increaser the included angle between two extension arms of the torsion spring. That is, the head bracing member 10 of the lightweight neck brace 1 can be unidirectionally adjusted with respect to the support member 20, that is to be adjusted unidirectionally in a direction away from the support member 20.

According to another aspect of the present invention, the present invention provides an adjustment method for the lightweight neck brace 1, wherein the adjustment method includes the following steps:

Switch the outer rotation member 31 and the inner rotation member 32 of the adjustment member 30 of the lightweight neck support 1 between the adjustment state and the free state, wherein the head bracing member 10 and the support member 20 of the lightweight neck support 1 are rotatably connected by means of the adjustment member 30, the head bracing member 10 is configured for bracing the head and is supported by the support member 20, and the support member 20 is configured for being supported on the trunk. When the inner rotation element 32 is engaged with the outer rotation element 31, the lightweight neck brace 1 is in the adjustment state. The head bracing member 20 of the lightweight neck brace 1 can be rotated in a direction away from the support member 20 unidirectionally but cannot be rotated towards the support member 20. When the inner rotation element 32 is disengaged from the outer rotation element 31, the lightweight neck brace 1 is in the free state, and the head bracing member 20 can be arbitrarily rotated towards or away from the support member 20.

According to one embodiment of the present invention, in the adjustment method, control the position of the inner rotating member 32 by the adjusting element 33, and the lightweight neck brace 1 can be switched from the free state to the adjustment state by pulling the adjusting element 33.

According to one embodiment of the present invention, in the adjustment method, control the position of the inner rotating member 32 by the adjusting element 33 and, when the adjusting element 33 is released, the lightweight neck brace 1 automatically switches from the free state to the adjustment state.

According to one embodiment of the present invention, in the adjustment method, massage the head by the head bracing member 10.

According to one embodiment of the present invention, in the adjustment method, change the included angle defined between the first bracing arm 11 and the second bracing arm 12 of the head bracing member 10 to fit different head sizes of the users.

According to another aspect of the present invention, the present invention provides an adjustment method for the lightweight neck brace 1, wherein the adjustment method includes the following steps:

Switch the outer rotation member 31 and the inner rotation member 32 of the adjustment member 30 to change positions of the head bracing member 10 and the support member 20 of the lightweight neck support 1 correspondingly, wherein the head bracing member 10 and the support member 20 of the light neck support 1 are rotatably connected by the adjustment member 30, the head bracing member 10 is configured for supporting the head and is supported on the support member 20, and the support member 20 is configured for being supported on the trunk. When the inner rotation element 32 is engaged with the outer rotation element 31, the lightweight neck brace 1 is in the adjustment state, and the head bracing member 20 of the lightweight neck brace 1 can be unidirectionally rotated in a direction away from the support member 20 but cannot be rotated toward the support member 20. When the inner rotation element 32 is disengaged from the outer rotation element 31, the lightweight neck brace 1 is in the free state, and the head bracing member 20 can be arbitrarily rotated toward or away from the support member 20.

According to one embodiment of the present invention, control the connecting arm 321 of the inner rotating member 32 by the adjusting element 33. In the adjustment state, the connecting arm 321 is controlled to be pressed against and engaged with the unidirectional tooth grooves 311 of the outer rotating member 31 that enables the outer rotating member 31 and the inner rotating member 32 to rotate with respect to each other unidirectionally. In the free state, the connecting arm 321 is controlled to be disengaged from the unidirectional tooth grooves 311 of the outer rotating member 31 that enables the outer rotating member 31 and the inner rotating member 32 to rotate freely.

According to one embodiment of the present invention, control the inner rotating member 32 by changing the position of the adjusting element 33. When the adjusting element 33 is in the first position, it is in the adjustment state. When the adjusting element 33 moves to the second position, the connecting arm 321 of the inner rotating member 32 is driven to disengage from the outer rotating member 31, so as to be in the free state.

According to one embodiment of the present invention, control the adjusting element 33 to control the inner rotating member 32 by pulling the head bracing member 10. When the head bracing member 10 is pulled outwards, the adjusting element 33 is driven to the first position and is in the adjustment state. When the force exerted by the head bracing member 10 on the adjusting element 33 is weakened or released, the adjusting element 33 can be reset to the first position.

According to one embodiment of the present invention, control the adjusting element 33 to control the inner rotating member 32 by pulling the support member 20. When the support member 20 is pulled outwards, the adjusting element 33 is driven to the first position, it is in the adjustment state. When the force exerted by the support member 20 on the adjusting element 33 is weakened or released, the adjusting element 33 can be reset to the first position.

According to one embodiment of the present invention, the adjusting element 33 located in the first position can be automatically reset to the second position, and thus driving the head bracing member 10 to reset to its original position.

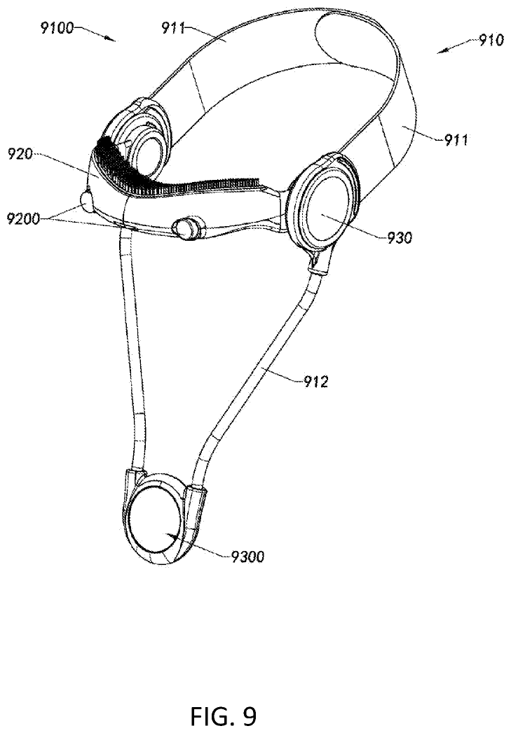

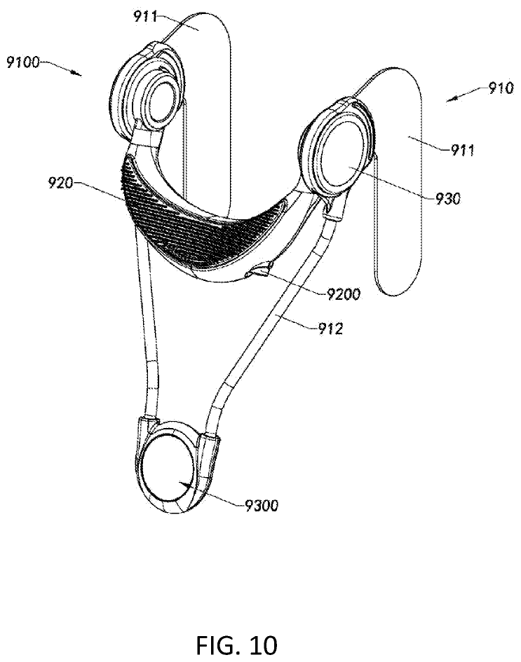

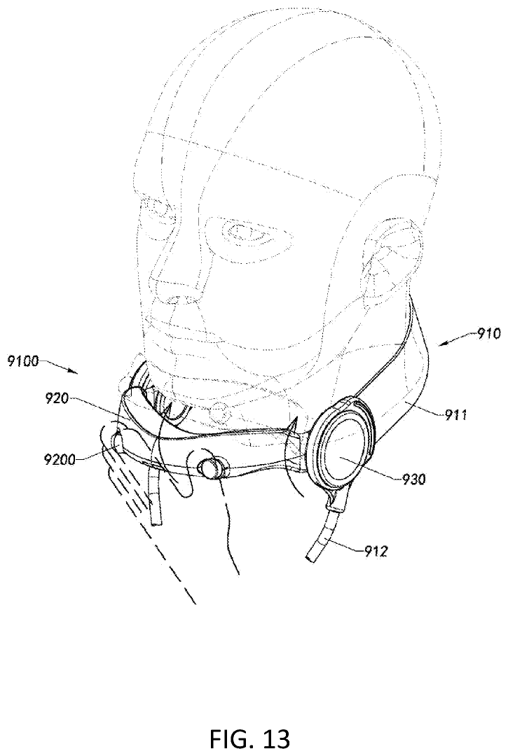

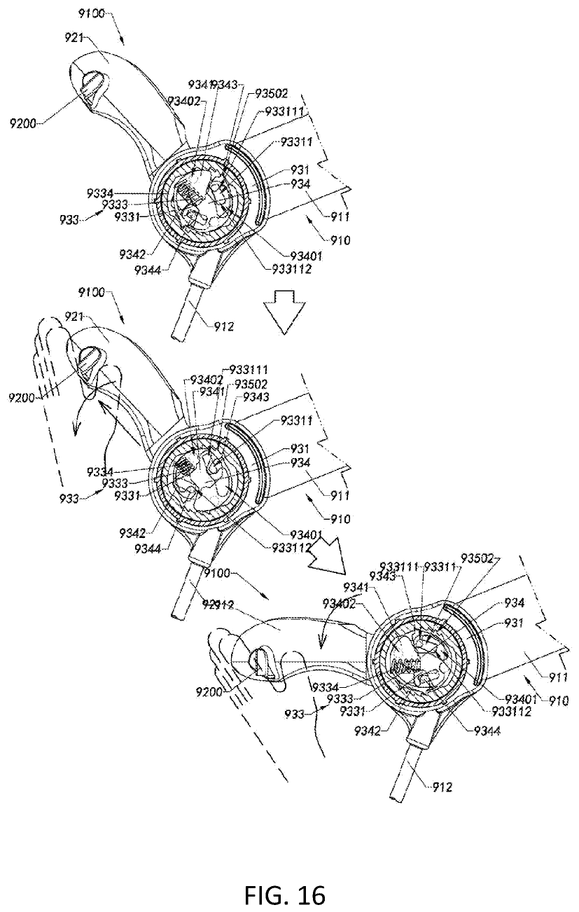

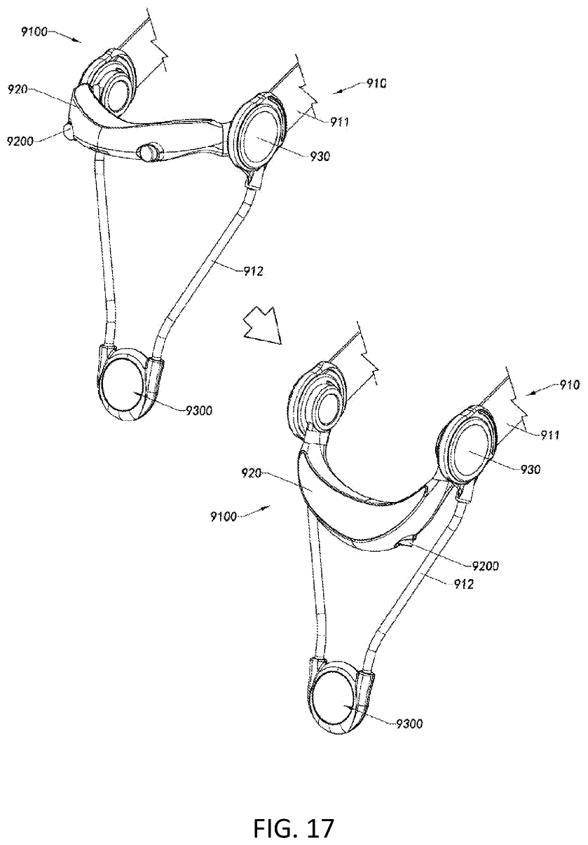

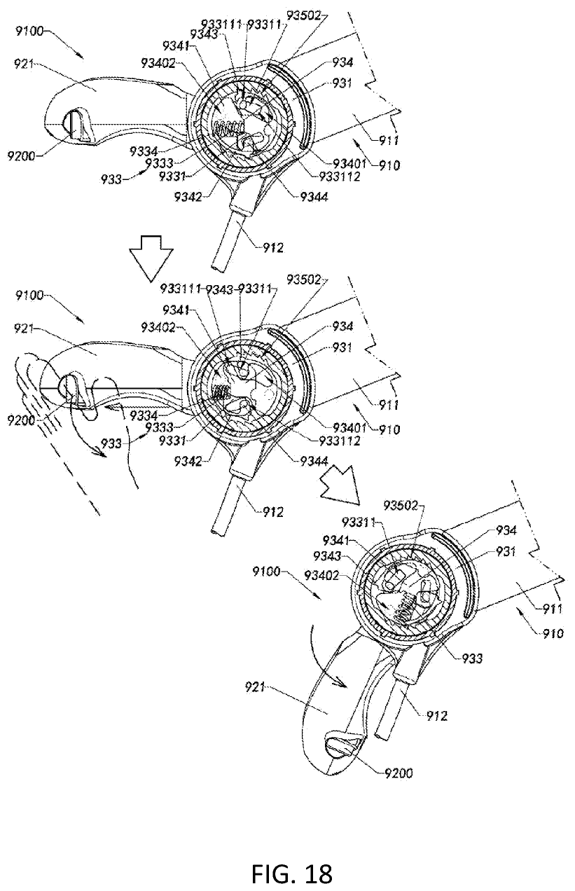

Referring to FIG. 9 to FIG. 18 of the drawings, a head support device 9100 according to the preferred embodiment of the present invention is illustrated and described in the following description, wherein the head support device 9100 can be worn on the shoulder and neck of the user to support the user's head so as to reduce the compression of the user's head on the cervical spine that facilitates the prevention and alleviation of cervical spine diseases.

Referring to FIG. 9 to FIG. 11, the head support device 9100 includes a wearing body 910 and a bracing body 920, wherein the bracing body 920 is rotatably connected to the wearing body 910, such that the head support 9100 can be switched between a unidirectional rotation state and a bidirectional rotation state. Also, when the head support device 9100 is in the unidirectional rotation state, the bracing body 920 is only allowed to be rotated upwards with respect to the wearing body 910, and when the head support 9100 is in the bidirectional rotation state, the bracing body 920 is allowed to rotate upwards and downwards with respect to the wearing body 910.

During the using of the head support device 9100 to support the head of the user, the head support device 9100 is in the unidirectional rotation state, so as to prevent the bracing body 920 from being rotated downward with respect to the wearing body 910 that may cause the bracing body 920 failing to always supporting the user's head upwards while the user's head from exerting downward force on the bracing body 920. If the user needs to adjust a wearing angle of the head support device 9100 or to fold up the head support device 9100, switch the head support device 9100 to the bidirectional rotation state, wherein by rotating the bracing body 920 upwards or downwards, the wearing angle of the head support device 9100 is adjustable or the head support device 9100 is folded up, such that the flexibility and practicability of the head support device 9100 are improved and different usage requirements of the users can be achieved.

Further, referring to FIG. 9 to FIG. 11, the head support device 9100 further includes two rotating mechanisms 930, wherein each of the rotating mechanisms 930 is connected between the wearing body 910 and the bracing body 920, such that the bracing body 920 is rotatably mount to the wearing body 910 by means of the rotating mechanisms 930, and that the head support device 9100 can be switched between the unidirectional rotation state and the bidirectional rotation state.

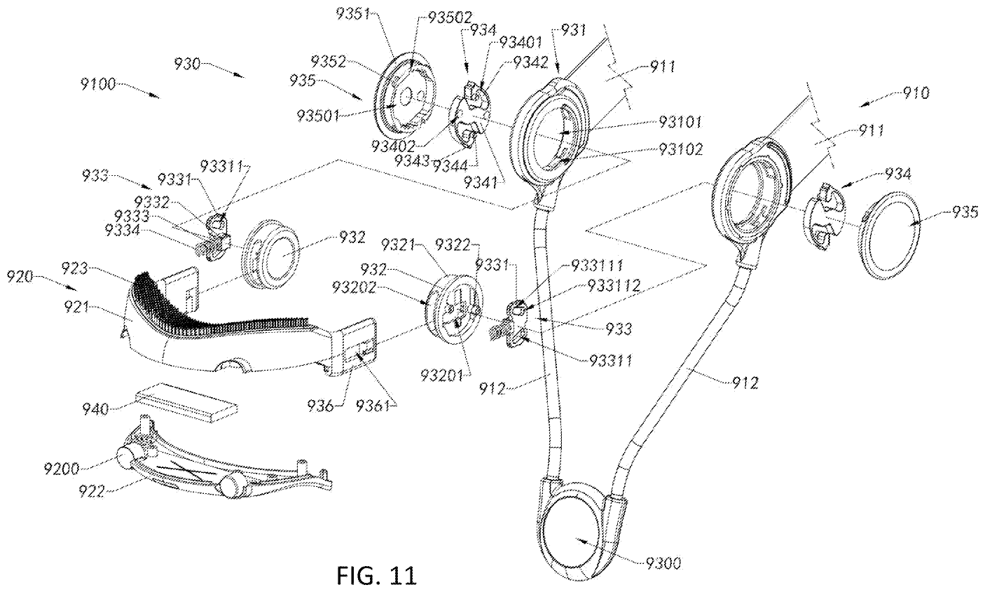

Referring to FIG. 11, each rotation mechanism 930 includes an assembly element 931, a rotation element 932, a driving element 933, a retaining element 934, a restriction element 935, and an operation element 936, wherein the assembly element 931 has an assembly chamber 93101 and at least two mounting grooves 93102 communicated with the assembly chamber 93101. The rotation element 932 has a receiving cavity 93201 and an assembly opening 93202 communicated with the receiving cavity 93201. The driving element 933 has two elongated restriction slots 93311. The retaining element 934 has two deformation spaces 93401 and two positioning grooves 93402. The restriction element 935 has an installation chamber 93501 and at least one set of restriction slant grooves 93502 communicated with the installation chamber 93501.

The rotation element 932 is rotatably mounted in the assembly chamber 93101 of the assembly element 931 in such a manner that an opening of the receiving cavity 93201 faces the assembly element 931, and that the assembly opening 9320 of the rotation element 932 is exposed on one side of the assembly element 931. The driving element 933 is installed in the receiving cavity 93201 of the rotation element 932, and the operation element 936 is installed in the assembly opening 932 of the rotation element 932 in a manner of being connected to the driving element 933 to avoid a separation of the rotation element 932 from the assembly element 931. Further, the driving element 933 can be rotated with respect to the assembly element 931 by operating the operation element 936.

The retaining element 934 is mounted to the driving element 933. The restriction element 935 is mounted to the assembly element 931. The restriction element 935 and the retaining element 934 are coupled with each other. Through the incorporation of the restriction element 935 and the retaining element 934, the operation element 936 allows the assembly element 931 and the restriction element 935 to rotate correspondingly. Also, the operation element 936 can be maintained at a predetermined angle. The wearing body 910 and the bracing body 920 are respectively connected to the assembly element 931 and the operation element 936. The user is able to operate the operation element 936 by applying a force on the bracing body 920 so as to adjust a relative angle between the operation element 936 and the wearing body 910.

Specifically, referring to FIG. 11, the retaining element 934 includes an installation base 9341, two cantilever arms 9342 and two retaining slant teeth 9343. The two cantilever arms 9342 are curvedly extended from the upper portion upwards and from the lower portion downwards respectively. A connecting end of each cantilever arm 9342 is integrally connected to the installation base 9341 and a free end of each cantilever arm 9342 is suspendingly retained on a respective side of the installation base 9341. Also, a deformation space 93401 is defined and formed between each of the cantilever arms 9342 and the installation base 9341, thereby allowing the cantilever arms 9342 to deform towards the installation base 9341. The retaining slant teeth 9343 extend outwardly from the cantilever arms 9342 respectively. The retaining slant teeth 9343 are respectively configured to be able to engage with the restriction slant grooves 93502 of the restriction element 935, such that the restriction element 935 can restrict a rotation of the retaining element 934 so as to enable the bracing body 920 being retained in a predetermined wearing angle.

The restriction element 935 includes a restricting portion 9351 and at least two installation protrusions 9352 extending from the restricting portion 9351. The installation chamber 93501 is formed in the restricting portion 9351. An inner wall of the restricting portion 9351 defining the installation chamber 93501 is recessed inwardly to form the restriction slant groove 93502. The installation protrusions 9352 of the restriction element 935 are configured to be engaged with the mounting grooves 93102 of the assembly element 931 respectively, so that the restriction element 935 is mounted to the assembly element 931. The retaining element 934 is installed in the restriction element 935 by engaging each of the retaining slant teeth 9343 with one of the restriction slant grooves 93502. The retaining slant teeth 9343 are meshed with the restriction portion 9351 of the restriction element 935, and the retaining slant teeth 9343 positioned in the restriction slant grooves 93502 of the restriction element 935 are only allowed to rotate in a one direction.

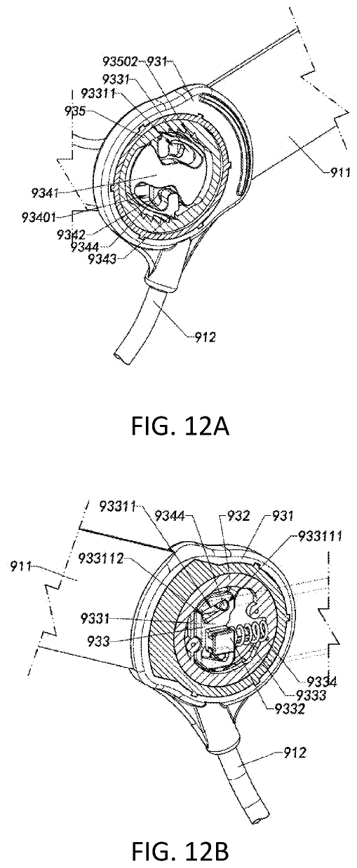

Further, referring to FIG. 12A to FIG. 14, each set of the restriction slant grooves 93502 allows the retaining slant teeth 9343 retained in the restriction slant grooves 93502 to move in only one direction. Specifically, the inner wall defining the restriction slant groove 93502 includes a long slant surface and a short slant surface, the long slant surface and the short slant surface are connected to each other to define the restriction slant groove 93502 with an acute angle. The long slant surfaces and the short slant surfaces of the adjacent restriction slant grooves 93502 are connected with each other, such that the short slant surface is configured to block a movement of the retaining slant tooth 9343 positioned in the respective restriction slant groove 93502 that the retaining slant tooth 9343 is only allowed to move along the long slant surface.

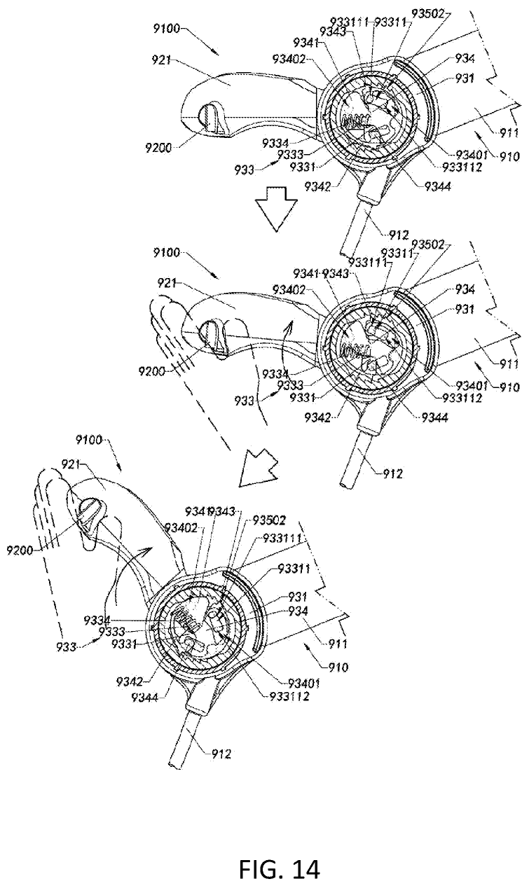

For example, referring to FIG. 14, each set of the restriction slant grooves 93502 is continuously distributed in a clockwise direction. In addition, in the clockwise direction, the short slant surface of a first one of the restriction slant grooves 93502 positioned at an upper portion of the restriction element 935 is close to the bracing body 920. The long slant surface of the first one of the set of restriction slant grooves 93502 positioned at the upper portion of the restriction element is away from the bracing body 920. The long slant surface of the last one of the set of restriction slant grooves 93502 positioned at a lower portion of the restriction element is close to the bracing body 920. The short slant surface of the last one of the set of restriction slant grooves 93502 positioned at the lower portion of the restriction element is away from the bracing body 920. Accordingly, the retaining slant teeth 9343 positioned in the restriction slant grooves 93502 respectively can only rotate in a clockwise direction, so as to allow the bracing body 920 of the head support device 9100 in a unidirectional rotation state to rotate in the clockwise direction. Person skilled in the art should understand that the above distribution of the restriction slant grooves 93502 is only an example, and the restriction slant grooves 93502 can be configured as long as restricting the retaining slant teeth 9343 to rotate unidirectional.

When the user wears the head support device 9100, the bracing body 920 is only allowed to rotate upwards that the retaining element 934 is driven to rotate counterclockwise with respect to the restriction element 935 and the retaining slant tooth 9343 moves counterclockwise into the adjacent restriction slant groove 93502. Therefore, the bracing body 920 is prevented from rotating downwards with respect to the wearing body 910 when a force is exerted to the bracing body 920 by the lowering head of the user so as to prevent the user from lowering his or her head involuntarily that, in turn, helps to prevent myopia. In other words, when the retaining slant teeth 9343 of the retaining element 934 are positioned in the respective restriction slant grooves 93502 of the restriction element 935, the head support device 9100 is in a unidirectional rotation state.

Preferably, the two sets of the restriction slant grooves 93502 are symmetrically distributed on an upper portion and a lower portion of the restriction element 935. Optionally, the two sets of the restriction slant grooves 93502 are able to be symmetrically distributed on a left portion and a right portion of the restriction element 935. Optionally, the two sets of the restriction slant grooves 93502 are also able to be distributed asymmetrically. It should be understood by those skilled in the art that the restriction slant grooves 93502 can also be implemented as one or more sets, and the corresponding retaining slant teeth 9343 can be implemented as one or more.

In addition, the specific number of each set of the restriction slant grooves 93502 is not limited. Referring to FIG. 12A and FIG. 12B, in the preferred embodiment of the present invention, each set of the restriction slant grooves 93502 includes five consecutively distributed restriction slant grooves 93502, that allows the bracing body 920 being retained at five different heights. That is to say, the head support device 9100 of the present invention not only can upwardly support the head of the user and fix a position of the head of the user, but also allow adjustment of different supporting angles, that limits the angle at which the user bows his head and reduces the pressure of the user's head on the cervical spine. Preferably, the head support device 9100 restricts the head of the user from lowering down to 60°. Optionally, the head support 9100 restricts the head of the user from lowering down to 45°. Optionally, the head support device 9100 restricts the head of the user head from lowering down to 30°. Optionally, the head support device 9100 restricts the head of the user from lowering down to 15°. Those skilled in the art should understand that the specific implementation of the restriction slant groove 93502 and the specific restriction on the lowering range of the user's head by the head support device 9100 are merely examples and cannot be used to limit the content and scope of the head supporting device 9100 described in the present invention.

Further, the rotation element 932 includes a rotating portion 9321 and two assembling pins 9322 extending outwardly from the rotating portion 9321. The receiving cavity 93201 and the assembly opening 93202 are formed in the rotating portion 9321. The assembling pins 9322 are distributed on two sides of the rotating portion 9321 respectively. The driving element 933 is assembled in the receiving cavity 93201 in a manner of being mounted on the operation element 936. The retaining element 934 is fixed to the rotation element 932 in such a manner that the two positioning grooves 93402 are respectively formed on two sides of the installation base 9341 corresponding to the two assembling pins 9322. When the operation element 936 drives the rotation element 932 to rotate with respect to the restriction element 935 and the assembly element 931, the two assembling pins 9322 of the rotation element 932 push the retaining element 934 to rotate synchronously.

Further, after the retaining slant teeth 9343 of the retaining element 934 are released from the restriction. That is, after the retaining slant teeth 9343 of the retaining element 934 are disengaged from the restriction slant grooves 93502 of the restriction element 935, the retaining element 934 is capable of rotating upwards and downwards with respect to the restriction element 935 and the assembly element 931.

The operation element 936 has an installation groove 9361 which is formed in an outer side of the operation element 936. The operation element 936 is inserted into the receiving cavity 93201 of the rotation element 932 through the assembly opening 93202 thereof. The operation element 936 is movably retained in the assembly opening 93202 of the rotation element 932 and the operation element 936 is able to move back and forth with respect to the rotation element 932.

The driving element 933 includes a driving portion 9331 and an assembly portion 9332, wherein the assembly portion 9332 is provided on one side of the driving portion 9331 and the assembly portion 9332 of the driving element 933 is installed in the installation groove 9361 of the operation element 936, such that the driving element 933 moves following the movement of the operation element 936. That is, the driving element 933 is movably retained in the receiving cavity 93201 of the rotation element 932 that the driving element 933 can move back and forth with respect to the rotation element 932.

The restriction slots 93311 of the driving element 933 are formed in the upper portion and the lower portion of the driving portion 9331 respectively, and that the two restriction slots 93311 are located above and below the assembly portion 9332 respectively. Correspondingly, the retaining element 934 further includes two driving posts 9344. The two driving posts 9344 are respectively extended outwardly from the free ends of the cantilever arms 9342 and positioned on outer sides of the retaining slant teeth 9343. The two driving posts 9344 of the retaining element 934 are respectively retained in the restriction slots 93311 of the driving element 933. The driving element 933 moves back and forth with respect to the restriction element 935 while following the movement of the operation element 936.

Further, referring to FIG. 11, the restriction slot 93311 of the driving element 933 has a first end 933111 and a second end 933112, wherein the first end 933111 and the second end 933112 of the restriction slot 93311 have a height difference. That is, the restriction slot 93311 of the driving element 933 extends inclinedly. Further, the two restriction slots 93311 are distributed in a “/\” shape in the driving portion 9331 of the driving element 933. That is, the second ends 933112 of the two restriction slots 93311 are close to each other while the first ends 933111 of the two restriction slots 93311 are away from each other.

When the retaining slant teeth 9343 of the retaining element 934 are positioned in the restriction slant grooves 93502 of the restriction element 935, the driving posts 9344 of the retaining element 934 are respectively positioned at the first ends 933111 of the restriction slots 93311. At this time, the head support device 9100 is in the unidirectional rotation state.

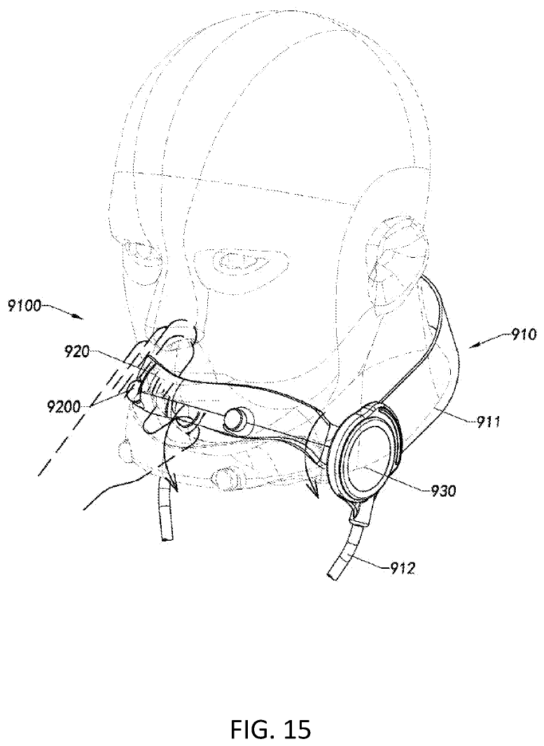

Referring to FIG. 15 to FIG. 18, while outwardly pulling the bracing body 920, the operation element 936 and the driving element 933 move outwards with respect to the retaining element 934. The two driving posts of the retaining element 934 gradually move outwards from the first end 933111 of the restriction slot 93311 towards the second end 933112. The two driving posts 9344 approach each other and the cantilever arms 9342 of the retaining element 934 deform towards the installation base 9341. The retaining slant teeth 9343 disengage with restriction slant grooves 93502 of the restriction element 935 and the rotation element 932 is allowed to rotate upward and downward with respect to the assembly element 931 and the restriction element 935. At this time, the head support device 9100 is switched to the bidirectional rotation state. After the usage of the head support device 9100 by the user, the user adjusts the head support device 9100 to the bidirectional rotation state and then folds the head support device 9100 downwards to reduce the overall size of the head support device 9100 for ease of storage and carrying.

After the bracing body 920 is adjusted to an appropriate angle, the bracing body 920 moves the operation element 936 and the driving element 933 inwards with respect to the retaining element 934. The two driving posts 9344 of the retaining element 934 gradually move from the second end 933112 of the restriction slot 93311 to the first end 933111 and the two driving posts 9344 move away from each other. The cantilever arms 9342 gradually return to their initial positions. The retaining slant teeth 9343 of the retaining element 934 enter in the respective restriction slant grooves 93502 of the restriction element 935 and thus the rotation element 932 is only allowed to rotate upwards with respect to the assembly element 931 and the restriction element 935. At this time, the head support device 9100 is switched from the bidirectional rotation state to the unidirectional rotation state.

In the preferred embodiment of the head support device 9100 according to the present invention, each driving element 933 of the rotating mechanism 930 further includes a retaining post 9333 and a resilient element 9334, wherein the retaining post 9333 is provided on one side of the driving portion 9331 and the assembly portion 9332 and the resilient element 9334 is provided along the retaining post 9333. The retaining post 9333 and the resilient element 9334 of the driving element 933 are installed in the receiving cavity 93201 of the driving element 933, wherein two ends of the resilient element 9334 abut against inner walls of the driving portion 9331 and the driving element 933 respectively. During a pulling the bracing body 920 outwards, the driving portion 9331 and the assembly portion 9332 of the driving element 933 follows the operation element 936 and move outwards, and the resilient element 9334 is compressed by the driving portion 9331 and the assembly portion 9332. When the bracing body 920 is rotated to an appropriate angle, the external force on the bracing body 920 is released, and the resilient element 9334 trends to restore to its initial position and drives the bracing body 920 to rapidly move inwardly, thereby the head restraint 9100 is rapidly switched from the bidirectional rotation state to the unidirectional rotation state. For example, but not limited to, the resilient element 9334 is implemented as a spring.

In the preferred embodiment of the head support device 9100 according to the present invention, the wearing body 910 includes two surrounding elements 911 and two supporting elements 912, wherein connection ends of the two surrounding elements 911 are respectively coupled to sides of the assembly elements 931 of the two rotating mechanisms 930 respectively, and the two supporting elements 912 are respectively extended downwardly from the assembly elements 931 of the two rotating mechanisms 930.

Two movable ends of the two surrounding elements 911 are capable of detachably connecting with each other so as to be convenient for user to wear. For example, but not limited to, the two surrounding elements 911 are detachably connected by means of snap connection, adhesive connection, or the like. The surrounding elements 911 are arranged to surround the neck of the user so as to maintain below the head of the user that facilitates the bracing body 920 to stably fit to the chin of the user to support the head of the user. Two ends of the two supporting elements 912 are connected to each other to form a V-shaped configuration such that the supporting elements 912 can be arranged to adaptive for being supported on the chest or the back of the user. The surrounding elements 911 and the supporting elements 912 cooperate with each other so that the head support device 9100 is capable of stably holding under the user's head.

Referring to FIG. 11, the bracing body 920 includes a bracing element 921, a shell 92 and a plurality of accommodating elements 923. The shell 922 is installed below the bracing element 921 and the plurality of accommodating elements 923 are upwardly and spacedly extended from the bracing element 921. The bracing element 921 is extended between the two operation elements 936 of the two rotating mechanisms 930. The accommodating elements 923 are made of soft and elastic material that can be deformed according to a contour of a chin of the user so as to fit the different chin contours of different users, thereby facilitating the bracing body 920 to support the head of the user by stably fitting with the chin of the user, which improves the flexibility and adaptability of the head support device 9100. In addition, during the using of the head support device 9100 by the user, the user's head exerts force on the accommodating elements 923 and the accommodating elements 923 deform and generate elastic reaction forces tending to reform and return to their original position, thereby upwardly pushing the head of the user to prevent the user's head from being lowered that facilitates the prevention of myopia.

The head support device 9100 further includes at least one distance detection member 9200, wherein the distance detection member 9200 is arranged on the bracing body 920 for detecting a distance between the head of the user and a target article, for example, but not limited to, detecting the distance between the user's head and a desktop, a book, a monitor, a television, and etc., so as to ensure that the user's head and the target article are kept in a healthy distance.

It is worth mentioning that the specific implementation of the distance detection member 9200 is not limited, wherein the distance detection body 9200 can be implemented as a camera device, a microwave detector, a distance sensor, or other distance detection devices known to those skilled in the art.

The head support device 9100 further includes a reminder member 9300, wherein the reminder member 9300 is communicatively connected with the distance detection member 9200. Once a distance between the head of the user and the target article is detected by the distance detection member 9200 to be smaller than a healthy distance, the reminding body 9300 reminds the user that facilitates the user to correct the sitting posture in time. In this way, bad habits that cause myopia, hunchback and the like can be effectively prevented.

Preferably, two distance detection members 9200 are implemented, and the two distance detection members 9200 are symmetrically arranged on two sides of the bracing body 920 respectively. The two distance detection members 9200 of the head support device 9100 worn under the user's head are located on both sides of the user's chin, such that the two distance detection members 9200 respectively detect distances between the two sides of the user's chin and the target article. For example, when the user wears the head support device 9100 while sitting and the two distance detection members 9200 detect that the distances between the user and the target article are the same, the user is in a proper sitting posture. When the user's cervical vertebra is inclined to one side, the head is also inclined to one side and the distances between the user's head and the target article detected by the two distance detection members 9200 are deviated. The reminder member 9300 is configured to timely remind the user to correct the sitting posture, so as to avoid a deformation of the spine caused by prolonged poor sitting posture.

It is worth mentioning that the specific implementation of the reminder member 9300 is not limited. The reminder member 9300 can be implemented as a reminder device through light, sound, vibration, or other reminder devices known to those skilled in the art. The embodiments of the distance detection member 9200 and the reminder member 9300 are only examples and cannot be a limitation on the content and scope of the head support device 9100 of the present invention.

The head support device 9100 further includes a power supply member 940, wherein the power supply member 940 is arranged and concealed between the bracing element 921 and the shell 922 and is electrically connected to the distance detection member 9200 and the reminder member 9300 to provide electric power to the distance measurement main body 9200 and the reminder member 9300.

It is worth mentioning that, referring to FIG. 9 to FIG. 18, the number of the rotating mechanism 930 is implemented as two, but in other examples of the present invention, the number of the rotating mechanism 930 can also be implemented as one. Specifically, one rotating mechanism 930 is connected with one end of the wearing body 910 and one end of the bracing body 920 allowing both unidirectional and bidirectional rotation, while the other end of the bracing body 10 is rotatably connected to the other end of the wearing body 910 in a bidirectional manner allowing bidirectional rotation. For example, but not limited to, one rotating mechanism 930 is connected to one end of the wearing body 910 and one end of the bracing body 920, while the other end of the wearing body 910 is rotatably connected to the other end of the wearing body 910 bidirectionally by means of gear connection. For example, when the rotating mechanism 930 is in the unidirectional rotation state and the bracing body 920 is rotated upwards with respect to the wearing body 910, the gear connection between the wearing body 910 and the bracing body 920 allows clockwise rotation correspondingly. When the rotating mechanism 930 is in the bidirectional rotation state and the bracing body 920 rotates upwards or downward with respect to the wearing body 910, the gear connection between the wearing body 910 and the wearing body 910 allows clockwise or counterclockwise rotation correspondingly. An included angle between the bracing body 920 and the wearing body 910 is allowed to be adjusted and the included angle between the bracing body 920 and the wearing body 910 can be retained and fixed too.

It should be understood by those skilled in the art that the embodiments of the present invention shown in the above description and the accompanying drawings are only examples and do not limit the present invention. The objects of the present invention have been fully and effectively achieved. The functional and structural principles of the present invention have been shown and described in the embodiments, and the embodiments of the present invention may be modified or modified in any way without departing from the principles.

One skilled in the art will understand that the embodiment of the present invention as shown in the drawings and described above is exemplary only and not intended to be limiting. The objects of the present invention have been fully and effectively achieved. The embodiments have been shown and described for the purposes of illustrating the functional and structural principles of the present invention and is subject to change without departure from such principles. Therefore, this invention includes all modifications encompassed within the spirit and scope of the following claims.

1: A lightweight neck brace, adapted for wearing on a torso of a user to support a head of the user, including:
a head bracing member configured for bracing the head at a predetermined position;
a support member which is coupled to the head bracing member and configured for being supported on the torso, wherein the head bracing member is supported by the support member so as for being supported by the torso; and
at least one adjustment member, wherein the at least one adjustment member is arranged at a connection position between the head bracing member and the support member and the head bracing member is connected with the support member by the at least one adjustment member in such a manner that the head bracing member is able to be folded towards the support member, so as to adjust an included angle defined between the head bracing member and the support member, wherein the lightweight neck support has an adjustment state, during the adjustment state, the head bracing member being allowed to be adjusted away from the support member unidirectionally.

2: The lightweight neck brace, as recited in claim 1, wherein the lightweight neck brace has a free state, and the lightweight neck brace is operable to switch between the adjustment state and the free state, wherein, in the free state, the head bracing member is allowed to be adjusted in a direction selected from a direction away from the support member and a direction toward the support member.

3: The lightweight neck brace, as recited in claim 1, wherein the at least one adjustment member includes an outer rotating member and an inner rotating member, wherein an inner wall of the outer rotating member defines a receiving cavity and the inner rotating member is accommodated in the receiving cavity, wherein the inner wall of the outer rotating member is recessed to form at least two unidirectional tooth grooves and the inner rotating member includes a base body and at least one connecting arm integrally extended from the base body outwardly in a suspended manner in such a manner that the at least one connecting arm is able to deform under an exerting force and return to an original form thereof after the exerting force is released, wherein the at least one connecting arm has a supporting surface formed and configured to be pressed against one of the at least two unidirectional tooth grooves to lock the outer rotating member and the inner rotating member, wherein in the adjustment state, the supporting surface of the at least one connecting arm is pressed and selectively abutted against one of the at least two unidirectional tooth grooves of the outer rotating member so as to restrict the inner rotating member to be able to merely rotate with respect to the outer rotating member unidirectionally.

4: The lightweight neck brace, as recited in claim 2, wherein the at least one adjustment member includes an outer rotating member and an inner rotating member, wherein an inner wall of the outer rotating member defines a receiving cavity and the inner rotating member is accommodated in the receiving cavity, wherein the inner wall of the outer rotating member is recessed to form at least two unidirectional tooth grooves and the inner rotating member includes a base body and at least one connecting arm integrally extended from the base body outwardly in a suspended manner in such a manner that the at least one connecting arm is able to deform under an exerting force and return to an original form thereof after the exerting force is released, wherein the at least one connecting arm has a supporting surface formed and configured to be pressed against one of the at least two unidirectional tooth grooves to lock the outer rotating member and the inner rotating member, wherein in the adjustment state, the at least one connecting arm is pressed and selectively abutted against one of the at least two unidirectional tooth grooves of the outer rotating member so as to restrict the inner rotating member to be able to merely rotate with respect to the outer rotating member unidirectionally, wherein in the free state, the at least one connecting arm of the inner rotating member is released from the at least two unidirectional tooth grooves so as to allow the inner rotating member to freely rotate with respect to the outer rotating member.

5-7. (canceled)

8: The lightweight neck brace, as recited in claim 4, wherein the at least one adjustment member further comprises an adjusting element which has at least one restriction slot, wherein the at least one connecting arm of the inner rotating member has a restricting pin configured to be extended to the restriction slot of the adjusting element so as to restrict the inner rotating member by the restricting element, wherein in the adjustment state, the restricting element presses the supporting surface of the at least one connecting arm against the one of the at least two unidirectional tooth grooves to restrict the outer rotating member and the inner rotating member to rotate unidirectionally, wherein in the free state, the restricting element restricts the at least one connecting arm to have a gap between the supporting surface thereof and the at least two unidirectional tooth grooves, such that the lightweight neck brace is able to switch between the adjustment state and the free state by adjusting the at least one adjustment member.

9: The lightweight neck brace, as recited in claim 2, wherein the at least one adjustment member includes an outer rotating member and an inner rotating member, wherein an inner wall of the outer rotating member defines a receiving cavity and the inner rotating member is accommodated in the receiving cavity, wherein the inner wall of the outer rotating member is recessed to form two sets of unidirectional tooth grooves and the inner rotating member includes a base body and two connecting arms integrally extended from two opposing sides of the base body outwardly in a suspended manner in such a manner that each of the two connecting arms are able to deform under an exerting force and return to an original form thereof after the exerting force is released, wherein the two connecting arms are configured to be pressed against two unidirectional tooth grooves of the two sets of unidirectional tooth grooves respectively to lock the outer rotating member and the inner rotating member, wherein in the adjustment state, the at least two connecting arms are pressed and selectively abutted against the two sets of unidirectional tooth grooves of the outer rotating member respectively so as to restrict the inner rotating member to be able to merely rotate with respect to the outer rotating member unidirectionally, wherein each of the two connecting arms of the inner rotating member forms a restricting pin, wherein the adjustment member further comprises an adjusting element which has two symmetrical restriction slots provided therein, a distance between the two restriction slots being arranged to gradually increase from one end to another end thereof, the restricting pins of the two connecting arms being restricted in the two restriction slots of the adjusting element respectively, wherein when the adjusting element is moved with respect to the inner rotating member and the restricting pins are moving from the ends of the two restriction slots having a maximum distance therebetween to the ends of the two restriction slots having a minimum distance therebetween, a distance between the two restricting pins is restricted by the two restriction slots of the adjusting element to decrease and the two connecting arms are driven by the adjusting element to move towards the base body and away from the two sets of unidirectional tooth grooves of the outer rotating member respectively, so that the lightweight neck brace is switched from the adjustment state to the free state.

10: The lightweight neck brace, as recited in claim 8, wherein the adjustment member further includes an adjustment member installation casing and a resilient connecting element, wherein the adjustment member installation casing forms an installation cavity therein and the adjusting element is positioned in the installation cavity, wherein the resilient connecting element is positioned between the adjusting element and the adjustment member installation casing, wherein the head bracing member is inserted in the adjustment member installation casing in a forth and back movable manner, wherein when the head bracing member is pulled outwards by an exerting force, the adjusting element is able to be driven to move outwards by the head bracing member, and the resilient connecting element is compressed at the same time, so as to disengage the connecting arm of the inner rotating member from the outer rotating member and switch to the free state, when the exerting force on the head bracing member is removed, the adjusting element returns to an original position thereof due to the resilient connecting element to switch to the adjustment state.

11: The lightweight neck brace, as recited in claim 9, wherein the adjustment member further includes an adjustment member installation casing and a resilient connecting element, wherein the adjustment member installation casing forms an installation cavity therein and the adjusting element is positioned in the installation cavity, wherein the resilient connecting element is positioned between the adjusting element and the adjustment member installation casing, wherein the head bracing member is inserted in the adjustment member installation casing in a forth and back movable manner, wherein when the head bracing member is pulled outwards by an exerting force, the adjusting element is able to be driven to move outwards by the head bracing member, and the resilient connecting element is compressed at the same time, so as to disengage the two connecting arms of the inner rotating member from the outer rotating member and switch to the free state, when the exerting force on the head bracing member is removed, the adjusting element returns to an original position thereof due to the resilient connecting element to switch to the adjustment state.

12: The lightweight neck brace, as recited in claim 10, further including a mounting member with an adjustable length, wherein the mounting member is extended between two ends of the head bracing member to form a size-adjustable wearing channel.

13: The lightweight neck brace, as recited in claim 11, wherein further including a mounting member with an adjustable length, wherein the mounting member is extended between two ends of the head bracing member to form a size-adjustable wearing channel.

14: The lightweight neck brace, as recited in claim 10, wherein the head bracing member includes a first bracing arm and a second bracing arm, wherein the first support arm is pivotably connected to the second bracing arm to be foldable toward the second bracing arm, wherein the support member comprises a first support arm and a second support arm, wherein the first support arm and the second support arm are respectively extended from two ends of the head bracing member outwardly until the first support arm and the second support arm are connected with each other to form a stable triangular support configuration, wherein a number of the adjustment member is two, wherein the two adjustment members are positioned at connecting positions of the head bracing member with the first support arm and the second support arm respectively, wherein thee first support arm of the support member is pivotally connected to the second support arm to be foldable towards the second support arm.

15: The lightweight neck brace, as recited in claim 11, wherein the head bracing member includes a first bracing arm and a second bracing arm, wherein the first support arm is pivotably connected to the second bracing arm to be foldable toward the second bracing arm, wherein the support member comprises a first support arm and a second support arm, wherein the first support arm and the second support arm are respectively extended from two ends of the head bracing member outwardly until the first support arm and the second support arm are connected with each other to form a stable triangular support configuration, wherein a number of the adjustment member is two, wherein the two adjustment members are positioned at connecting positions of the head bracing member with the first support arm and the second support arm respectively, wherein thee first support arm of the support member is pivotally connected to the second support arm to be foldable towards the second support arm.

16-30. (canceled)

31: The lightweight neck brace, as recited in claim 2, further including a second adjustment member, wherein the at least one adjustment member pivotally connects a first end of the head bracing member with a first end of the support member and the second adjustment member pivotally connects a second end of the head bracing member with a second end of the support member.

32: The lightweight neck brace, as recited in claim 31, wherein each of the at least one adjustment member and the second adjustment member includes an outer rotating member and an inner rotating member, wherein the outer rotating member is connected to the head bracing member and the inner rotating member is connected to the support member.

33: The lightweight neck brace, as recited in claim 31, wherein each of the at least one adjustment member and the second adjustment member includes an outer rotating member and an inner rotating member, wherein the inner rotating member is connected to the head bracing member and the outer rotating member is connected to the support member.

34: The lightweight neck brace, as recited in claim 32, wherein an inner wall of the outer rotating member defines a receiving cavity and the inner rotating member is accommodated in the receiving cavity, wherein the inner wall of the outer rotating member is recessed to form at least two unidirectional tooth grooves and the inner rotating member includes a base body and at least one connecting arm integrally extended from the base body outwardly in a suspended manner in such a manner that the at least one connecting arm is able to deform under an exerting force and return to an original form thereof after the exerting force is released, wherein the at least one connecting arm has a supporting surface formed and configured to be pressed against one of the at least two unidirectional tooth grooves to lock the outer rotating member and the inner rotating member, wherein in the adjustment state, the at least one connecting arm is pressed and selectively abutted against one of the at least two unidirectional tooth grooves of the outer rotating member so as to restrict the inner rotating member to be able to merely rotate with respect to the outer rotating member unidirectionally, wherein in the free state, the at least one connecting arm of the inner rotating member is released from the at least two unidirectional tooth grooves so as to allow the inner rotating member to freely rotate with respect to the outer rotating member.

35: The lightweight neck brace, as recited in claim 33, wherein an inner wall of the outer rotating member defines a receiving cavity and the inner rotating member is accommodated in the receiving cavity, wherein the inner wall of the outer rotating member is recessed to form at least two unidirectional tooth grooves and the inner rotating member includes a base body and at least one connecting arm integrally extended from the base body outwardly in a suspended manner in such a manner that the at least one connecting arm is able to deform under an exerting force and return to an original form thereof after the exerting force is released, wherein the at least one connecting arm has a supporting surface formed and configured to be pressed against one of the at least two unidirectional tooth grooves to lock the outer rotating member and the inner rotating member, wherein in the adjustment state, the at least one connecting arm is pressed and selectively abutted against one of the at least two unidirectional tooth grooves of the outer rotating member so as to restrict the inner rotating member to be able to merely rotate with respect to the outer rotating member unidirectionally, wherein in the free state, the at least one connecting arm of the inner rotating member is released from the at least two unidirectional tooth grooves so as to allow the inner rotating member to freely rotate with respect to the outer rotating member.

36: The lightweight neck brace, as recited in claim 32, wherein an inner wall of the outer rotating member defines a receiving cavity and the inner rotating member is accommodated in the receiving cavity, wherein the inner wall of the outer rotating member is recessed to form two sets of unidirectional tooth grooves and the inner rotating member includes a base body and two connecting arms integrally extended from two opposing sides of the base body outwardly in a suspended manner in such a manner that each of the two connecting arms are able to deform under an exerting force and return to an original form thereof after the exerting force is released, wherein the two connecting arms are configured to be pressed against two unidirectional tooth grooves of the two sets of unidirectional tooth grooves respectively to lock the outer rotating member and the inner rotating member, wherein in the adjustment state, the at least two connecting arms are pressed and selectively abutted against the two sets of unidirectional tooth grooves of the outer rotating member respectively so as to restrict the inner rotating member to be able to merely rotate with respect to the outer rotating member unidirectionally, wherein each of the two connecting arms of the inner rotating member forms a restricting pin, wherein the adjustment member further comprises an adjusting element which has two symmetrical restriction slots provided therein, a distance between the two restriction slots being arranged to gradually increase from one end to another end thereof, the restricting pins of the two connecting arms being restricted in the two restriction slots of the adjusting element respectively, wherein when the adjusting element is moved with respect to the inner rotating member and the restricting pins are moving from the ends of the two restriction slots having a maximum distance therebetween to the ends of the two restriction slots having a minimum distance therebetween, a distance between the two restricting pins is restricted by the two restriction slots of the adjusting element to decrease and the two connecting arms are driven by the adjusting element to move towards the base body and away from the two sets of unidirectional tooth grooves of the outer rotating member respectively, so that the lightweight neck brace is switched from the adjustment state to the free state.

37: The lightweight neck brace, as recited in claim 33, wherein an inner wall of the outer rotating member defines a receiving cavity and the inner rotating member is accommodated in the receiving cavity, wherein the inner wall of the outer rotating member is recessed to form two sets of unidirectional tooth grooves and the inner rotating member includes a base body and two connecting arms integrally extended from two opposing sides of the base body outwardly in a suspended manner in such a manner that each of the two connecting arms are able to deform under an exerting force and return to an original form thereof after the exerting force is released, wherein the two connecting arms are configured to be pressed against two unidirectional tooth grooves of the two sets of unidirectional tooth grooves respectively to lock the outer rotating member and the inner rotating member, wherein in the adjustment state, the at least two connecting arms are pressed and selectively abutted against the two sets of unidirectional tooth grooves of the outer rotating member respectively so as to restrict the inner rotating member to be able to merely rotate with respect to the outer rotating member unidirectionally, wherein each of the two connecting arms of the inner rotating member forms a restricting pin, wherein the adjustment member further comprises an adjusting element which has two symmetrical restriction slots provided therein, a distance between the two restriction slots being arranged to gradually increase from one end to another end thereof, the restricting pins of the two connecting arms being restricted in the two restriction slots of the adjusting element respectively, wherein when the adjusting element is moved with respect to the inner rotating member and the restricting pins are moving from the ends of the two restriction slots having a maximum distance therebetween to the ends of the two restriction slots having a minimum distance therebetween, a distance between the two restricting pins is restricted by the two restriction slots of the adjusting element to decrease and the two connecting arms are driven by the adjusting element to move towards the base body and away from the two sets of unidirectional tooth grooves of the outer rotating member respectively, so that the lightweight neck brace is switched from the adjustment state to the free state.

38: The lightweight neck brace, as recited in claim 37, further including a mounting member with an adjustable length, wherein the mounting member is extended between two ends of the head bracing member to form a size-adjustable wearing channel.
